SEQUENCE LISTING (13 sequences)

SEQ ID NO: 1 - protein, 1091 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 2 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 4 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 5 - protein, 583 aa, Homo sapiens
MNLLPPKPKYNPLRNESLSSMEEGASGSTPPEELPSPPASSLGPILPPLPGDDSPTTLCSFFPRMSNLRLANPAGGRPGSKGEPGRAADDGEGIVGAAMPDSGPLPLLQDMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 6 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 7 - protein, 168 aa, Homo sapiens
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 8 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 9 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 10 - protein, 118 aa, Homo sapiens
MSGGSSCSQTPSRAIPATRRVVLGDGVQLPPGDYSTTPGGTLFSTTPGGTRIIYDRKFLMECRNSPVTKTPPRDLPTIPGVTSPSSDEPPMEASQSHLRNSPEDKRAGGEESQFEMDI

SEQ ID NO: 11 - protein, 525 aa, Homo sapiens
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 12 - protein, 217 aa, Homo sapiens [sequence omitted]

SEQ ID NO: 13 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

(19) United States
(12) Patent Application Publication
(10) Pub. No.: US 2009/0275546 A1
(43) Pub. Date: Nov. 5, 2009

(54) Diagnostic tests and personalized treatment regimes for cancer stem cells

(75) Inventors: Michele Signore, Rome (IT); Ruggero De Maria, Rome (IT); Lance A. Liotta, Bethesd, MD (US); Emanuel F. Petricoin, Gainesville, VA (US)

Correspondence Address: QUINE INTELLECTUAL PROPERTY LAW GROUP, P.C., P O BOX 458, ALAMEDA, CA, 94501, US

(73) Assignees: Istituto Superiore di Sanita, Roma (IT); George Mason University, Fairfax, VA (US)

(21) Appl. No.: 12/386,002

(22) Filed: Apr. 10, 2009

Related U.S. Application Data
(60) Provisional application No. 61/123,970, filed on Apr. 10, 2008.

(51) Int. Cl. A61K 31/395; C40B 30/06; C12N 5/02

(52) U.S. Cl. 514/183; 506/10; 435/375

(57) ABSTRACT

Provided are methods of identifying a metabolic target in a cancer stem cell that include using a microarray to identify intracellular signaling networks within a population of cancer stem cells that respond to a growth factor for the stem cell. Also provided are methods of determining a personalized therapeutic regime that include receiving metabolic information relating to a cancer stem cell in a patient, determining the patient's personal criteria relevant to the therapeutic regime, and combining the metabolic and personal criteria. Also provided are a diagnostic test for establishing a personalized therapeutic regime for a colon cancer patient and methods of reducing colon cancer stem cells/treating colon cancer.

### CROSS-REFERENCE TO RELATED APPLICATIONS

The present application claims priority to and benefit of U.S. Provisional Patent Application No. 61/123,970, entitled DIAGNOSTIC TEST AND PERSONALIZED TREATMENT REGIMES FOR CANCER STEM CELLS, by Signore et al., filed Apr. 10, 2008. This prior application is incorporated herein by reference in its entirety for all purposes.

### FIELD OF THE INVENTION

The present invention relates to methods for identifying cancerous cells, particularly cancer stem cells (CSCs).

### BACKGROUND OF THE INVENTION

It has been demonstrated that cancer is a robust system and is resistant to chemotherapy and radiotherapy due to its capacity to withstand external perturbations through genetic instability and cellular heterogeneity [19]. The use of novel targeted anticancer agents, alone or in combination with chemotherapy has significantly improved cancer treatment and management [20,21,22,23]. However, cancer is able to escape even highly focused treatments, as demonstrated by the emergence of Imatinib-resistant clones in CML [24]. An urgent clinical goal is to identify functionally important molecular signaling networks associated with subpopulations of patients that do not respond to conventional combination chemotherapy.

Unique molecular signatures define different tumor types and patients subsets. Mapping of protein signaling networks associated with specific cancer stem cell (CSC) populations can be useful in identifying new targets for therapy. What are needed in the art are methods of identifying metabolic targets in CSCs and methods of using such metabolic targets in determining personalized therapeutic regimes to treat cancer. These and other needs are provided by the invention described herein, as will be apparent upon reading the following disclosure.

### SUMMARY OF THE INVENTION

The phosphorylation or activation state of kinase-driven signaling networks can provide information that can be useful in the diagnosis, treatment, and predicting a prognosis for cancer. The methods of the invention are generally directed to identifying metabolic targets of intracellular signaling networks in cancer stem cells, determining a personalized therapeutic regime for a patient based on metabolic information relating to the cancer stem cell, and administering the regime to the patient to treat cancer.

Thus, in a first aspect, the invention provides methods for identifying a metabolic target in a cancer stem cell, which methods include using a protein microarray, e.g., a reverse phase protein microarray, to identify intracellular signaling networks within a population of cancer stem cells that respond to a growth factor for the stem cell. Optionally, the population of cancer stem cells can be sampled from a patient or subject diagnosed with cancer or from a patient or subject who has not previously been diagnosed with cancer, as part of a cancer screening program.

In some embodiments of the methods, the growth factor to which the signaling networks respond is EGF, e.g., wherein the EGF is capable of activating an EGF Receptor (EGFR). In certain embodiments, the cancer stem cells can show EGF-R activation or Bcl-2 hyper-phosphorylation. In other embodiments of the methods, the cancer stem cells show hyper-phosphorylated p38MAPK, NF-κB and Shc, which is indicative of Melanoma cancer stem cells. Alternatively or additionally, the cancer stem cells can optionally show enhanced HER2 signaling, which is indicative of colon cancer stem cells; or mTOR pathway hyper-activation or hyper-phosphorylated Bad levels, which is indicative of lung cancer stem cells. Some cancer stem cells screened by the methods can optionally show both mTOR and EGF-R hyper-phosphorylation, low EGF-R and high mTOR and GSK3-betalevels, or high phospho-Bad and phospho-Adducin levels, which is indicative of glioblastoma cancer stem cells.

Once a metabolic target in a cancer stem cell has been identified, the methods can optionally include identifying suitable treatments for a patient and administering the treatments to the patient, so as to provide a personalized treatment program based on the metabolic target. Once a metabolic target or pattern of targets has been identified by the identification of the signaling networks, the methods can optionally include cross-referencing the target or pattern of targets with a database of targets or target patterns.

Relatedly, the invention provides a method for identifying a metabolic target in a colon cancer stem cell. The method includes using a protein microarray to identify intracellular signaling networks within a population of cancer stem cells that respond to a growth factor for the stem cell.

The invention also provides a method of treating a patient with cancer that include identifying a metabolic target in a cancer stem cell, using a protein microarray to identify intracellular signaling networks within a population of cancer stem cells that respond to a growth factor for the stem cell, and treating the cancer by administering a therapeutic agent directed at said metabolic target. In a related aspect, the invention provides methods of determining a personalized therapeutic regime. These methods include receiving metabolic information relating to a cancer stem cell from a patient; determining at least one metabolic target criterion in said cancer stem cell, receiving personal information relating to the patient, determining personal criteria relevant to the personalized therapeutic regime using the personal information, and combining the at least one metabolic target criteria and the personal criteria to determine the personalized therapeutic regime for the patient.

Optionally, the methods of determining a personalized therapeutic regime can include identifying multiple targets, leading to the establishment of a pattern of targets that comprise the levels of a particular metabolite and/or the variation of the metabolite over time. Optionally, a metabolite can form part of at least one pathway and the personalized therapeutic regime can target the at least one pathway. The personalized therapeutic regime that is determined in the methods can optionally include single active agents or combinations of active agents that target single or multiple pathways. The active agents can optionally be selected from compounds, such as pharmaceuticals, antibodies, and RNAi, for instance, and combinations thereof.

The personal information that can be used to determine the personal therapeutic regime can optionally include: genomic criteria, proteomic criteria, biochemical criteria, metabolomic criteria; the patient's sex, the patient's age, the patient's gender, the patient's current medication, the patient's past medication, the patient's family medical history, the patient's personal medical history, and the patient's lifestyle. Additionally or alternatively, the personal information can optionally include any one or combination of the following: the patient's ethnicity, the patient's weight, the patient's Body Mass Index, incidence of a condition of potential interest for the personalized therapeutic regime in the patient's family, and the patient's environmental conditions. The patient's personal information can optionally be obtained in the form of a questionnaire provided to the user over a communications network. The patient's genomic, proteomic, biochemical or metabolomic information can optionally obtained from analyzing a sample from the patient. Analyses that can be performed on the sample can include any one or a combination of the following: genotyping; haplotyping; analysis of the patient's RNA; analysis of the patient's proteome; and analysis of the patient's metabolome.

Once the therapeutic regime for the patient has been determined, the methods of determining a personalised therapeutic regime can optionally include administering the therapeutic regime to the patient. The methods can optionally include receiving feedback information patient related to the effects of the personalised therapeutic regime from the patient. Optionally, the methods can further include using the feedback to determine an updated personalised therapeutic regime according to the any effects of the personalised therapeutic regime on the patient.

The invention provides methods of identifying a cancer stem cell, which methods include assaying for activation of the EGF Receptor (EGFR) in a sample of cells, wherein the presence of activated EGFR or phosphorylation of Bcl2 being indicative of a cell in said sample being a cancer stem cell. The sample of cells that is assayed can optionally comprise cancerous cells, stem cells or a mixture of both. In other embodiments, the sample of cells can comprise non-cancerous stem cells, allowing identification and targeting or separation of the Cancer Stem Cells (CSCs) from the remaining cells.

A method of facilitating proliferation of colon cancer stem cells is also provided by the invention. The method comprises contacting said cells with one or more inhibitors of p38MAPK, c-Raf and/or PKA/ROCK. The invention also provides a method of reducing the number of colon cancer stem cells in a population of said cells, comprising contacting said cells with geldanamycin (or its analogues, such as 17-aag or 17-dmag) and, optionally, with one or more inhibitors of inhibitors of PKC, p70S6K, Akt and/or MEK1. Relatedly, the invention provides a method of treating colon cancer in a patient, comprising administering geldanamycin17-aag or 17-dmag, and, optionally, one or more inhibitors of p38MAPK, c-Raf and/or PKA/ROCK to said patient.

The invention provides a diagnostic test for establishing a personalized treatment regime for a colon cancer patient, which test includes conducting microarray analysis on cancer stem cells to predict pathway activation linked to colon cancer, probing of isolated colon cancer stem cells with inhibitors, to validate a predicted pathway activation, and combining pathway activation data with inhibitor data to determine individualized therapies for said patient.

One of skill in the art will appreciate that the methods of the invention can be used alone or in combination with one another.

Kits that permit a practitioner to use the methods described herein, e.g., to identify a metabolic target in a cancer stem cell, to determine a personalized therapeutic regime, etc., also a feature of this invention. The kits can include a reverse phase protein microarray, reagents for use with a protein microarray, and/or the like. The kits can also include additional useful reagents, such as antibodies, buffers, and the like. Such kits also typically include, e.g., instructions for use of the compounds and other reagents, e.g., to practice the methods of the invention, as well as any packaging materials for packaging the components of the kits.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a metabolic target” includes a combination of two or more metabolic targets; reference to “cancer stem cells” includes mixtures of cancer stem cells, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

Cancer stem cells: As used herein, “Cancer stem cells” or “CSCs” are cancer cells, e.g., found within tumors, that possess characteristics associated with normal stem cells, e.g., the ability to give rise to all cell types found in a particular cancer sample. CSCs are tumorigenic and can generate tumors through the stem cell processes of self-renewal and differentiation into multiple cell types. CSCs are proposed to persist in tumors as a distinct population contribute to relapse and/or metastasis.

Metabolic target: As used herein, a “metabolic target” refers to a biological molecule, e.g., a protein, whose, e.g., expression level, phosphorylation state, etc., can be evaluated as an indicator of disease progression and/or of a pharmacological response to therapeutic treatment.

### BRIEF DESCRIPTION OF THE DRAWINGS

FIG. 1 shows score plots of Principal Component Analyses (PCA) on starvation experiment.

FIG. 2 shows hierarchical Clustering (HCL) of CSCs cultured in the presence of growth factors.

FIG. 3 shows score plots of Principal Component Analyses (PCA) on differentiation experiment.

FIG. 4 shows hierarchical Clustering of CSCs before and after induction of differentiative program.

FIG. 5 shows drug screening experimental settings.

FIG. 6 shows means and 95% confidence intervals of normalized results obtained from three independent experiments on four different colon CSC lines are plotted against kinase inhibitors.

FIG. 7 shows hierarchical clustering of statistical significance (p values) for each kinase inhibitor over the colon CSCs. Inhibitors are labeled on the right with the name of the targeted protein kinase activity. N.S. states for non-significant.

FIG. 8 is a point chart of the results from the titration experiment on various colon CSCs. Normalized viability triplicates are plotted against inhibitors within three different concentration levels.

FIG. 9 is a point chart of the results from dose-response analysis on four colon CSCs. Normalized viability (5 replicates) is plotted against escalating doses of TRAIL, 17-AAG and 17-DMAG.

FIG. 10 shows 20× Phase contrast images of colon CSCs untreated or cultivated 48 h in the presence of 62 ng/mL TRAIL.

FIG. 11 is a point chart of the drug combination experiment on CTSC#85 (TRAIL-resistant colon CSC line). Normalized viability at 24 h is plotted against drug combinations. The synergistic effect is highlighted by the centermost arrow for “TRAIL IC50/17-AAG IC50.”

### DETAILED DESCRIPTION

CSCs derived from colon and lung carcinomas, glioblastomas, and melanomas were subjected to RPPMA analysis upon starvation or treatment with stimuli that induce cell differentiation or apoptosis.

Surprisingly, we found that CSCs derived from all the tumors examined share EGF-R (Epidermal Growth Factor Receptor) activation but, depending on their origin, they also have unique signatures: melanoma-derived CSCs display p38MAPK, NF-KB and Shc activation, while colon-derived CSCs show high levels of HER2 signaling. Glioblastoma-derived stem cells have hyper-active mTOR pathway and GSK30 or ErbB3 activation.

Modulators and, especially, inhibitors of colon cancer stem cell growth and proliferation are also provided, as are methods of treatment of colon cancer.

In the past two years there has been growing support for the cancer stem cell (CSC) theory. When first proposed by Cohnheim in 1875 [2], the CSC concept was intended to describe tumorigenesis as a derangement of normal stem cells from their physiological behavior [3]. According to such hypothesis it has been recently published that Lgr5 is expressed in colon cancer cells [4] potentially explaining the origin of colon cancer as a malignant evolution of normal Lgr5-expressing colonic stem cells [5]. However, to date it has not been definitively demonstrated that CSCs derive from normal stem cells that undergo a transformation process.

After the discovery of leukemia stem cells by Bonnet and Dick [6], the CSCs properties were defined as the ability of single clones to self-renew and recapitulate the original tumor in mouse xenograft models. The modern significance of cancer stem cell refers therefore to their functional properties rather than their origin [7]. Cancer stem cells have been identified from a variety of solid tumors, strongly supporting the CSC theory as a key mechanism underlying tumorigenesis. CD 133 was used as a marker to isolate brain [8], colon [9,10] and lung [11] tumor stem cells. Putative prostate cancer stem cells have been shown to possess a CD44⁺/α₂β₁^{hi}/CD133⁺ phenotype [12] while prospective identification of breast cancer stem cells has been performed through the use of CD24 and CD44 markers [13]. Pancreatic tumor stem cells have been identified surface expression of CD24, CD44 ad ESA [14] and melanoma stem cells have been recently isolated by selection for the ABCB5 chemoresistance-related protein [15]. Dynamics studies indicate the existence of a rare population of cells with stem-cell-like properties also in CML (Chronic Myeloid Leukemia) [16]. A list of the markers through which different types of cancer cells with stem cell properties have been identified, is well reviewed in [17].

The greatest implication of the CSC model is the molecular characterization of CSCs in quest of a definitive targeted therapy [18]. It has been demonstrated that cancer is a robust system and is resistant to chemotherapy and radiotherapy due to its capacity to withstand external perturbations through genetic instability and cellular heterogeneity [19]. The use of novel targeted anticancer agents, alone or in combination with chemotherapy has significantly improved cancer treatment and management [20,21,22,23]. However, cancer is able to escape even highly focused treatments as demonstrated by the emergence of Imatinib-resistant clones in CML [24]. Unique molecular signatures define different tumor types and patients subsets.

Surprisingly, we have discovered that all CSC types share EGF-R activation and Bcl-2 hyper-phosphorylation. Without being bound by theory, it is thought that phosphorylation of Bcl-2 inactivates its anti-apoptotic function only after JNK activation.

We also found that melanoma CSCs have hyper-phosphorylated p38MAPK, NF-KB and Shc, colon CSCs have enhanced HER2 signaling, and that two out of three lung CSCs have mTOR pathway hyper-activation. Another one lung CSC had hyper-phosphorylated Bad levels. Furthermore, glioblastoma CSCs have both mTOR and EGF-R hyper-phosphorylation or low EGF-R and high mTOR and GSK3-beta levels. One glioblastoma CSC co-clustered with a melanoma CSC by high phospho-Bad and phospho-Adducin levels.

Thus, in a first aspect, the present invention provides a method for identifying a metabolic target in a cancer stem cell, comprising the use of a protein microarray to identify intracellular signaling networks within a population of cancer stem cells that respond to a growth factor for the stem cell.

In one embodiment, the protein microarray is a reverse phase protein microarray. In one embodiment, the growth factor is EGF. In one embodiment, the EGF is as defined below. In one embodiment, the EGF is capable of activating the EGF Receptor (EGFR).

The population of cancer stem cells can be sampled from a patient or subject diagnosed with cancer or from a patient or subject who has not previously been diagnosed with cancer, e.g., as part of a screening program.

Once the metabolic target has been identified, suitable treatments for the patient can identified and administered to the patient. Thus, a personalized treatment program, e.g., specific for the patient, can be based on the metabolic target identified.

The same applies for more than one target, so that increasing levels or layers of personalization or personal specification can be devised and instigated. Equally, once a particular target or pattern of targets has been identified, this can be cross-referenced with a database of targets or target patterns.

The present invention provides a method of treating a patient with cancer by identifying a metabolic target in a cancer stem cell, using a protein microarray to identify intracellular signaling networks within a population of cancer stem cells that respond to a growth factor for the stem cell, and treating the cancer by administering a therapeutic agent directed at said metabolic target.

Also provided is a method of determining a personalized therapeutic regime that comprises receiving metabolic information relating to a cancer stem cell patient, determining at least one metabolic target criterion in said cancer stem cell, receiving personal information relating to the patient, determining personal criteria relevant to the personalized therapeutic regime using the personal information, and combining the at least one metabolic target criterion and the personal criteria to determine the personalized therapeutic regime for the patient.

The personal information can include genomic, proteomic, biochemical or metabolomic criteria or information about the sex, age, gender, current or past medication, family and personal medical history and/or lifestyle of the patient.

In one embodiment, multiple targets can be identified. In one embodiment, this leads to the establishment of a pattern of targets, which pattern can comprise both the levels of a particular metabolite and/or the variation of the metabolite over time.

In one embodiment, the personal information comprises any one or combination of the following: the ethnicity of the patient; the sex of the patient, the weight of the patient, the Body Mass Index of the patient, age of the patient, the incidence of a condition of potential interest for the personalized therapeutic regime in the patient's family, and the environmental conditions of the patient.

In one embodiment, the personal information is obtained in the form of a questionnaire. The questionnaire can be provided to the user over a communications network. In one embodiment, genomic, proteomic, biochemical or metabolomic information is obtained from analysis of a sample from the patient. The analysis can comprise any of or a combination of the following: genotyping; haplotyping, analysis of the patient's RNA, analysis of the patient's proteome, and/or analysis of the patient's metabolome.

In one embodiment, once the therapeutic regime for the patient has been determined, the personalized therapeutic regime is administered to the patient. Once the personalized therapeutic regime has been administered to the patient, feedback information can be received from the patient related to the effects of the personalized therapeutic regime.

In one embodiment, the method can further comprise using the feedback information to determine an updated personalized therapeutic regime according to the effects of the personalized therapeutic regime on the patient. In one embodiment, the method further comprises administering the updated personalized therapeutic regime to the patient. Once the therapeutic regime has been administered, augmentation information can be provided to the patient in order augment the personalized therapeutic regime.

In one embodiment, the metabolite forms part of at least one pathway and the treatment method targets said at least one pathway. In one embodiment, there can therefore be single or multiple pathways each or all of which can be targeted by single active agents or combinations of active agents.

The active agents can be selected from compounds, such as pharmaceuticals, antibodies, and RNAi, for instance. Any combinations thereof are also envisaged.

In a further aspect, the present invention provides a method of identifying a cancer stem cell that comprises assaying for activation of the EGF receptor (EGFR) in a sample of cells, the presence of activated EGFR and/or phosphorylation of Bcl2 being indicative of said cell being a cancer stem cell.

The criteria for defining a cell clone as a cancer stem cell are established in the literature, of course.

The protein sequence for EGFR is provided in SEQ ID NO: 1, below.

Preferably, the sample of cells comprises cancerous cells, stem cells or a mixture of both. Preferably, the sample of cells comprises non-cancerous stem cells, allowing identification and targeting or separation of the CSCs from the remaining cells.

In one embodiment, the samples comprise cancerous cells only, derived from tumor specimens. In one embodiment, CSCs are sleeted for in an in vitro serum-free culture system. In one embodiment, it is not necessary to separate the CSCs from other cells, so long as they can be identified or behave as CSCs, in that they clonally self-renew and are able to generate mouse xenografts comparable to the original tumor.

Preferably, activated EGFR is detected on the surface of the cell, as EGFR is normally a cell surface receptor.

In one embodiment, EGFR is detected on sample buffer lysates where proteins are denatured. In one embodiment, the protein array is part of a is a cell-free assay system. In one embodiment, the array is a reverse-phase protein microarray.

The epidermal growth factor receptor (EGFR; ErbB-1; HER1 in humans) is the cell-surface receptor for members of the epidermal growth factor family (EGF-family) of extracellular protein ligands. The epidermal growth factor receptor is a member of the ErbB family of receptors, a subfamily of four closely related receptor tyrosine kinases: EGFR (ErbB-1), BER2/c-neu (ErbB-2), Her 3 (ErbB-3) and Her 4 (ErbB-4).

EGFR (epidermal growth factor receptor) normally exists on the cell surface and is activated by binding of its specific ligands, including epidermal growth factor and transforming growth factor α (TGPα). ErbB2 has no known direct activating ligand, and can be in an activated state constitutively or become active upon heterodimerization with other family members such as EGFR.

Upon activation by its growth factor ligands, EGFR undergoes a transition from an inactive monomeric form to an active homodimer, although there is some evidence that preformed inactive dimers can also exist before ligand binding. In addition to forming homodimers after ligand binding, EGFR can pair with another member of the ErbB receptor family, such as ErbB2/Her2/neu, to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein-tyrosine kinase activity. As a result, autophosphorylation of several tyrosine (Y) residues in the C-terminal domain of EGFR occurs. These are Y845, Y992, Y1045, Y1068, Y1148 and Y1173 (see SEQ ID NO: 1).

This autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to DNA synthesis and cell proliferation. Such proteins modulate phenotypes such as cell migration, adhesion, and proliferation. The kinase domain of EGFR can also cross-phosphorylate tyrosine residues of other receptors it is aggregated with, and can itself be activated in that manner.

Thus, it is preferred that the activated EGFR of the present invention can detected by the presence of the active homodimer for of EGFR. Preferably, the activity of the homodimer can be assessed or measured by determining its kinase activity. This can be achieved by measuring phosphorylation, namely the transfer of phosphate groups from high-energy donor molecules, such as ATP to specific target molecules (substrates).

Activation can be direct phosphorylation by EGFR or by downstream activated kinases. In this later case the substrate for phosphorylation by the activated EGFR can be Bcl2. After EGF-R activation Bcl-2 is phosphorylated by downstream activated kinases.

The protein sequence for Bcl2 is provides in SEQ ID NO: 2, below.

Preferably, the Bcl2 is hyper-phosphorylated.

Also provided is a method for assaying for the presence of a cancer stem cell comprising identifying CSCs with activated EGFR and/or phosphorylated BcL2, preferably hyper-phosphorylated BcL2.

The proteins listed in Table 2 are useful and preferred indicia of CSCs, particularly those that are significantly up- or down-regulated in CSCs compared to normal cells.

Preferably, the CSC is an epithelial CSC, preferably a melanoma CSC. Preferably said CSC has or displays at least one of: hyper-phosphorylated p38MAPK, NF-κB and Shc.

SEQ ID NO: 3: p38MAPK PROTEIN SEQUENCE ACCESS NUMBER: NP_{—}001306.1

SEQ ID NO: 4: NF-κB (p65) PROTEIN SEQUENCE ACCESS NUMBER:

SEQ ID NO: 5: p66 Shc PROTEIN SEQUENCE ACCESS NUMBER: NP_{—}892113

This comparison on tissue sections can be achieved by microdissection.

Alternatively, the CSC is preferably a gastrointestinal CSC, preferably a colon CSC. Preferably said CSC has or displays enhanced HER2 signaling.

SEQ ID NO: 6: HER2 PROTEIN SEQUENCE ACCESS NUMBER: AAA75493

Alternatively, the CSC is preferably a respiratory CSC, e.g., preferably a lung CSC. Preferably said CSC has or displays mTOR pathway hyper-activation or hyper-phosphorylated Bad levels.

SEQ ID NO: 7: Bad PROTEIN SEQUENCE ACCESS NUMBER: 092934

Alternatively, the CSC is preferably a nervous system CSC, preferably a central nervous system CSC, preferably a brain tumor CSC, and most preferably a glioblastoma (especially glioblastoma multiforme) CSC. Preferably said CSC has or displays at least one of: both mTOR and EGF-R hyper-phosphorylation or low EGF-R and high mTOR and GSK3-β levels. Alternatively, said CSC can display high phospho-Bad and phospho-Adducin levels.

SEQ ID NO: 8: mTOR PROTEIN SEQUENCE ACCESS NUMBER: NP_{—}004949

SEQ ID NO: 9: GSK3-β PROTEIN SEQUENCE ACCESS NUMBER: NP_{—}002084.2

Where reference is made to a CSC being of or from a particular cell, tissue, organ or system type, then it will be understood that this means that the CSC is derived from said cell, tissue, organ, or system and cannot become a stem cell for another cell, tissue, organ, or system type. Suitable cell surface markers and cell morphology will accompany certain cell, tissue, organ, or system types and can be identifiable by the skilled person.

Analysis of the phosphorylation of the proteome was important in the present invention.

In some embodiments, the majority of the phosphorylation seen is due to activation of EGFR. Table 1 below shows a large number of phosphorylated proteins and the phosphorylated residues. Some or all of these phosphorylation events can be mediated by EGFR directly, and there can be a cascade effect.

Without being bound by theory, this could probably be as these cells are cultured in the presence of EGF, the ligand of EGF-R, but CSCs could have other intrinsic activation signatures. The presence of high doses of EGF in the culture medium seems to negatively regulate the phosphorylation of some EGF-R signaling kinases like Akt or ERK.

We also induced differentiation in CSCs, which we found, surprisingly, to lead to a general down-modulation of the endpoints in a heat map, for instance as shown herein, after differentiation induction. Further analysis showed tumor-wise clustering of differentiation as described below.

As explained in Example 1, differentiation can be induced by withdrawal of growth factors and, in some embodiments, addition of serum can induce CSC differentiation.

Glioblastoma CSCs were found to lose mTOR pathway activation but maintain phospho-EGF-R Y1045 and phosho-Bcl-2 S70 upon differentiation.

After differentiation, melanoma CSCs display high levels of 4EBP1, p70S6K and mTOR phosphorylation.

Upon differentiation, three out of four lung-derived CSCs show high pospho-eIF4E S209 levels.

Although only one colon carcinoma CSC cell line was included in the differentiation experiments, after its differentiation, we observed an up-regulation of phospho-Bcl-2 S70 that can be confirmed in further experiments.

Induction of differentiation is important in the treatment of CSCs, as it has the same therapeutic effect as killing the cancer cells, i.e. the cells lose their proliferative potential and, therefore, induction of differentiation can be used in the prophylaxis and treatment of cancer targeted at said CSCs.

Thus, in a further aspect, the present invention provides a method of inducing differentiation in a CSC by withdrawal of growth factors and, in some embodiments, addition of serum. In some embodiments, induction of CSC differentiation is achieved by starving the cell of growth factors or by removing growth factors, for instance using antibodies or RNAi.

Also provided is a method of identifying a differentiated cancer stem cell, comprising assaying for the presence of absence of phosphorylation as particular site on a protein, the presence or absence of phosphorylation being indicative of said cell being a differentiated cancer stem cell.

Preferably, the CSC is an epithelial CSC, preferably a melanoma CSC. Preferably said differentiated CSC has or displays at least one of: high levels of 4EBP1, p70S6K and mTOR phosphorylation. We have shown that high levels of certain phosphorylated endpoints in certain CSC samples. Therefore, we propose a wide range of targets for the same lesions, to be used for therapeutic intervention.

SEQ ID NO: 10: 4EBP1 PROTEIN SEQUENCE ACCESS NUMBER: NP_{—}004086.1

SEQ ID NO: 11: p70S6K PROTEIN SEQUENCE ACCESS NUMBER: NP_{—}003152.1

Alternatively, the differentiated CSC is preferably a gastrointestinal CSC, preferably a colon CSC. Preferably said CSC has or displays phospho-Bcl-2 S70, preferably up-regulation thereof. Preferably said CSC comprises Bcl2 phosphorylated at position S70.

Alternatively, the differentiated CSC is preferably a respiratory CSC, preferably a lung CSC. Preferably said CSC has or displays high pospho-eIF4E S209 levels. Preferably said CSC comprises eEF4E phosphorylated at position S209.

SEQ ID NO: 12: eIF4E PROTEIN SEQUENCE ACCESS NUMBER: NP_{—}001959.1

Alternatively, the differentiated CSC is preferably a nervous system CSC, preferably a central nervous system CSC, preferably a brain tumor CSC and most preferably a glioblastoma (especially glioblastoma multiforme) CSC. Preferably said CSC has or displays little or no MTOR pathway activation and preferably has or displays phospho-EGF-R Y1045 and/or phosho-Bcl-2 S70. Preferably said CSC comprises EGF-R phosphorylated at position Y1045 and/or Bcl2 phosphorylated at position S70.

Table 4 shows the in CSCs after differentiation induction as compared to CSCs cultured in the presence of growth factors. The proteins listed as being statistically significant, by increase or decrease compare to un-induced control cells, are preferred indicia that differentiation has been initiated and preferably fully induced.

Most preferably, reverse phase protein microarray analysis can be used in aspects of the invention to determine changes in the activation, phosphorylation, and so forth of the protein, receptor, or pathway, for instance. Preferably, the changes indicative of CSCs or differentiated CSCs are measurable by reverse phase protein microarray analysis.

Alternatives to this measuring system can include western blot or flow cytometry. In some embodiments, high-throughput is preferred, and it is much less powerful and sensitive.

CSCs represent a small population within the tumor compartment. CSCs are endowed with self-renewal capabilities and are able, through asymmetric division, to generate a heterogeneous population of cancer cells, thus sustaining tumor growth and progression [1]. The CSC concept has tremendous implications for cancer therapy, eventually leading to treatments based on specific targeting of CSCs as CSCs are responsible for tumor growth and relapse.

We took advantage of reverse phase protein microarray technology, an established high-throughput protein quantification platform [27,28], and phosphoproteomic analysis to study and map the signaling networks and pathways of CSCs. We evaluated the activation and/or expression of proteins involved in proliferation, differentiation and survival pathways in CSCs obtained from several patients.

CSCs derived from colon and lung carcinomas, glioblastomas, and melanomas were subjected to RPPMA analysis upon starvation or treatment with stimuli that induce cell differentiation or apoptosis.

Surprisingly, we found that CSCs derived from all the tumors examined share EGF-R (Epidermal Growth Factor Receptor) activation, but, depending on their origin, they also have unique signatures. For example, melanoma-derived CSCs display p38MAPK, NF-KB and Shc activation, while colon-derived CSCs show high levels of HER2 signaling. Glioblastoma-derived stem-cells have hyper-active mTOR pathway and GSK3β or ErbB3 activation.

SEQ ID NO: 13: ErbB3 PROTEIN SEQUENCE ACCESS NUMBER: NP_{—}001973.2

Differentiative stimuli induced significant changes in the majority of the endpoints evaluated in glioblastoma stem cells, whereas few changes were observed after induction of differentiation in lung or melanoma stem cells.

In a further aspect, the invention provides a method for identifying a metabolic target in a colon cancer stem cell. The method comprises using a protein microarray to identify intracellular signaling networks within a population of cancer stem cells that respond to a growth factor for the stem cell. Suitable targets include those described further below.

We also conducted further analysis on colon cancer stem cells. We showed that a combination of certain inhibitors with geldanamycin could provide a cocktail of effective drugs for colon cancer therapy. Thus, RPPMA-guided pathway analysis is useful, for individualized patient treatment.

What is surprising is that some inhibitors lead to significant proliferation, revealing that cancer cells have complex circuitry where some pathways can be unblocked by micro-environmental clues leading to cell proliferation. Such pro-survival compounds are inhibitors of p38MAPK, c-Raf or PKA/ROCK (NCBI gene IDs are 1432, 5894 and 5566/6093 respectively, incorporated herein by reference). Specifically, these enzyme inhibitors are SB 202190 (p38MAPK), ZM 336372 (c-Raf) and HA-1077 (PKA/ROCK). The respective Pubchem compound IDs are: SB 202190: CID 5353940; ZM336372: CID 9549283: and HA-1077: CID 3547.

Accordingly, the invention also provides a method of facilitating proliferation of colon cancer stem cells comprising contacting said cells with one or more inhibitors of p38MAPK, c-Raf and/or PKA/ROCK.

Such agents can be added singly or in combination to the culture medium, for instance, to enhance stem cells cloning efficiency.

On the other hand, drugs that significantly lead to a reduction in colon CSC numbers are inhibitors of PKC, ERK2, p70S6K, Akt, MEK1, and PDGFRK (NCBI gene EDs 5578, 5594, 6198, 207, 5604 and 5159 respectively, incorporated herein by reference). Specifically, these enzyme inhibitors are Ro-31-8220 (PKC), Rottlerin (PKC), 5-Iodotubercidin (ERK2), Rapamycin (p70S6K), Triciribine (Akt), U-0126 (MEK1) and AG17 (PDGFRK). Ro-31-8220: CID 5083. The respective Pubchem compound IDs are: Rottlerin: CID 5281847; 5-Iodotubercidin: CID 1830; Rapamycin: CID 5284616; Triciribine: CID 65399; U0126: CID 3006531; AG17 (Tyrphostin A9): CID 126295.

Accordingly, the invention also provides a method of reducing the number of colon cancer stem cells in a population of said cells, which method comprises contacting said cells with geldanamycin (or its analogues, such as 17-aag or 17-dmag) and, optionally, with one or more inhibitors of inhibitors of PKC, p70S6K, Akt and/or MEK1. Said method can be in vitro or can be in vivo and, hence, part of a method for treating colon cancer.

Accordingly, the invention also provides a method of treating colon cancer in a patient, which method comprises administering geldanamycin (or its analogues, such as 17-aag, e.g., 17-Allylamino-17-demethoxygeldanamycin, or 17-dmag, e.g., 17-dimethylaminoethylamino-17-demethoxy-geldanamycin), and, optionally, one or more inhibitors of p38MAPK, c-Raf and/or PKA/ROCK to said patient. The inhibitors can be delivered in the form of peptides/proteins in a pharmaceutically acceptable form and dose. Nucleotides encoding said peptides or protein in a suitable delivery vehicle, such as a viral vector (e.g. adeno- or lenti-viral), preferably under the control of a suitable promoter, are also envisaged in some embodiments. However, it will also be appreciated that, as some of these inhibitors can be small molecules, they can also be administered via liposomes or carrier proteins.

The combination both geldanamycin and the inhibitors is useful here, but it will be appreciated that, as with all combinations above, the timing of administration of the geldanamycin (or its analogues, such as 17-aag or 17-dmag) and the one or more inhibitors can be together or, most preferably, separately. In some embodiments, geldanamycin (or its analogue) can be administered before treatment with the one or more inhibitors. This can provide a more efficacious treatment, but this can depend on the ladme (pharmacokinetic) profile of the drugs, as will be apparent to the skilled physician.

Alternatively, a combination of TRAIL and geldanamycin (or its analogues, such as 17-aag or 17-dmag) is effective to induce apoptosis in colon cancer stem cells. The combination of these two drugs had already been proposed for colon cancer, but it was done on commercially available cell lines (ref #8, below). However, no one has thought to use these on cancer stem cell lines.

It is noteworthy that there is a functional relationship between TRAIL and geldanamycin, in that, although they differ in structure, they are known to act in part on the same pathway (NF-κB). Thus, in some embodiments, TRAIL and/or geldanamycin can be used interchangeably or even replaced with other modulators, preferably inhibitors, of the NF-κB pathway.

Where reference is made above to geldanamycin, it will be appreciated that this also includes its analogues (and visa versa), including 17-aag/17-dmag where geldanamycin is derivatized at its 17 amino acid position to aag or dmag. Geldanamycin and its analogues are discussed in U.S. Pat. No. 6,890,917, incorporated herein by reference. Geldanamycin is a benzoquinone ansamycin antibiotic, and it was originally found to be a fermentation product of Streptomyces hygroscopicus. Thus, in some embodiments, Geldanamycin can be replaced with another suitable benzoquinone ansamycin antibiotic.

This new data is complementary to the work done in Example 1 with reverse phase protein microarrays (RPPMAs). By using inhibitors, we “probed” cancer stem cells for their molecular activation status, in a process called “pathway hunting”.

Accordingly, the invention also provides a diagnostic test for establishing a personalized treatment regime for a colon cancer patient, comprising:
1—conducting microarray analysis on cancer stem cells to predict pathway activation linked to colon cancer;
2—probing of isolated colon cancer stem cells with inhibitors, for instance those mentioned above, to thereby validate the predicted pathway activation; and
3—combining pathway activation data inhibitor data to thereby determine individualized therapies for said patient.

For instance, RPPMA analysis can first be performed on tissue from the patient, for instance both laser-capture microdissected cells from a patient's tumor and on cancer stem cells obtained from the very same patient. These cancer stem cells can then be treated with the present inhibitors and a responsiveness/sensitivity chart is thereby generated. Finally, RPPMA pathway activation profiles, matched with the data of responsiveness/sensitivity to inhibitors, will allow the selection of inhibitors that will be effective for in vivo use. For example, if from RPPMA analysis mTOR and Akt pathways are activated and cells are sensitive to Rapamycin and Triciribine inhibitors, a combinatorial treatment with these inhibitors or their analogues, would be envisioned.

The isolation of the cancer stem cells from tumor specimens from the patient can also form part of this test, although it will be appreciated that this step can occur same time in advance and the cells or tissue can then be stored, for instance.

Modulators and, especially, inhibitors of colon cancer stem cell growth and proliferation are therefore also provided, as are methods of treatment of colon cancer comprising the same.

All references cited herein are herby incorporated by reference, unless otherwise apparent.

The present invention will now be illustrated with reference to the following Examples and Figures.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Identifying Cancer Stem Cell Biomarkers

### Stem Cell Culture and Treatment

Tumor samples were obtained in accordance with consent procedures approved by the Internal Review Board of Department of Laboratory Medicine and Pathology, Sant'Andrea Hospital, University La Sapienza, Rome and of the Institute of Pathological Anatomy, Catholic University of Rome. Surgical specimens were washed several times with PBS (phosphate buffered saline) without calcium and magnesium and incubated overnight in DMEM-F12 medium supplemented with high doses of penicillin/streptomycin and amphotericin B to avoid contamination. Tissue dissociation was carried out by mechanical dissociation for glioblastoma or enzymatic digestion (20 mg/ml collagenase II, Gibco-Invitrogen, Carlsbad, Calif.) for 2 h at 37° C. for colon, melanoma and lung tumor specimens. Recovered cells were cultured at clonal density in serum-free medium containing 50 mg/ml insulin, 100 mg/ml apo-transferrin, 10 mg/ml putrescine, 0.03 mM sodium selenite, 2 mM progesterone, 0.6% glucose, 5 mM HEPES, 0.1% sodium bicarbonate, 0.4% BSA (Bovine Serum Albumin), glutamine and antibiotics, dissolved in DMEM-F12 medium (Invitrogen, Carlsbad, Calif., USA) and supplemented with 20 ng/ml EGF (Epidermal Growth Factor) and 10 ng/ml bFGF (basic Fibroblast Growth Factor).

Ultra-low Attachment flasks (Corning, N.Y., USA) non-treated for tissue culture were used to reduce cell adherence and support growth of undifferentiated tumor spheres. The medium was replaced or supplemented with fresh growth factors twice a week until cells started to form floating aggregates. Cultures were expanded by mechanical dissociation of spheres, followed by re-plating of both single cells and residual small aggregates in fresh medium containing EGF and bFGF and denoted as complete medium. For growth factor starvation experiments, 5×10⁵ cells were washed twice with culture medium (devoid of EGF and bFGF), and were resuspended in culture medium in the presence or in the absence of growth factors. After 6 days cells were washed in PBS and cell pellets were promptly frozen with liquid nitrogen. For differentiation experiments 5×10⁵ cells were washed twice with growth factor-free medium and were replated in the presence of growth factors or of 5% FBS (Fetal Bovine Serum).

After 14 days, cells were washed twice in PBS and cell pellets were promptly frozen with liquid nitrogen. All cell pellets were stored for a maximum time of two weeks at −80° C. before being lysed.

### Reverse Phase Protein Microarray

Cell pellets were lysed in T-PER (Pierce, Rockford, Ill., USA) buffer additioned of 100 mM Sodium Orthovanadate, 200 mM PEFABLOC (Roche, Base1, Switzerland), 5 mg/ml Aprotinin, 1 mg/ml Pepstatin-A and 5 mg/ml Leupeptin (Sigma-Aldrich, St. Louis, Mo., USA). Lysates were diluted with 2× Tris-Glycine SDS Sample Buffer (Invitrogen, Carlsbad, Calif., USA) prior printing on nitrocellulose slides (Whatman, Maidstone, Kent, UK) and were spotted in duplicate with the Aushon 2470 contact pin arrayer (Aushon BioSystems Inc., Billerica, Mass., USA), either in 5-point dilution curves or just in undiluted-1:4 pairs, thus assuring that the linear detection range was encompassed for the chosen antibody concentration. Positive and negative expression control lysates were printed on every slide in a ten-point two-fold dilution curve, comprising A431+/−EGF, HeLa +/−Pervanadate (Becton Dickinson, Franklin Lakes, N.J., USA) and Jurkat +/−FasL (Cell Signaling Technology Inc., Danvers, Mass., USA).

Array staining with antibodies was carried out on an automated slide stainer accordingly to manufacturer's instructions (Autostainer CSA kit, DAKO, Carpinteria, Calif.). Each slide was incubated with a single primary antibody at room temperature for 30 min. The negative control slide was incubated with antibody diluents alone. Biotinylated secondary antibody was either goat anti-rabbit IgG H+L (1:5000; Vector Labs, Burlingame, Calif.) or anti-mouse (1:10; from CSA kit). Streptavidin-conjugated IRDye680® (LI-COR Biosciences, Lincoln, Nebr., USA) was used as a final signal generating step.

All antibodies used in these studies were validated for specificity by immunoblotting prior to use on the arrays. All endpoints tested in these studies and the companies from which antibodies were bought are listed in Table 1.

Table 1 shows the endpoints (proteins and phosphorylated proteins) assayed in the paper. The alphanumerical code indicates the amino acid site of phosphorylation (e.g. Y705 means tyrosine 705).

TABLE 1
Endpoints

| TOTAL | PHOSPHORYLATED |
|---|---|
| 4EBP1 * | phospho-4EBP1 S65 * |
| Akt * | phospho-4EBP1 T70 * |
| BAD * | phospho-Adducin S663 * |
| Bax * | phospho-Akt S473 * |
| Bcl-XL * | phospho-Akt T308 * |
| β-Actin * | phospho-Bad S112 * |
| β-Catenin * | phospho-Bad S136 * |
| Caspase 3 * | phospho-Bad S155 * |
| Caspase 8 * | phospho-Bcl2 S70 * |
| Caspase 9 * | phospho-Bcl-2 T56 * |
| CD133 ^{‡} | phospho-βCatenin T41-S45 * |
| c-Kit ^{&} | phospho-c-abl T735 * |
| Cleaved Caspase3 D175 * | phospho-c-abl Y345 * |
| Cleaved Caspase9 D315 * | phospho-c-Kit Y703 ** |
| CREB * | phospho-c-Raf S338 * |
| Cyclin A * | phospho-CREB S133 * |
| Cyclin D1 ^{†} | phospho-EGF-R Y1045 * |
| Cyclin E ^{†} | phospho-EGF-R Y1068 * |
| EGF-R * | phospho-EGF-R Y1148 ^{&} |
| EGF-R L858R * | phospho-EGF-R Y1173 ^{&} |
| ErbB3 * | phospho-EGF-R Y992 * |
| ERK * | phospho-eIF4E S209 * |
| GSK-3β * | phospho-eIF4G S1108 * |
| Her-2 ^{#} | phospho-eNOS S1177 * |
| HIF1-α ^{†} | phospho-erbB2 Y1248 ^{%} |
| HSP90 * | phospho-erbB3 Y1289 * |
| IkB-α * | phospho-ERK T202/Y204 * |
| IRS-1 * | phospho-Estrogen Rec-α S118 * |
| MEK * | phospho-FADD S194 * |
| mTOR * | phospho-FAK Y397 ^{†} |
| NF-κB * | phospho-FAK Y576/77 * |
| p27-Kip1 ^{†} | phospho-FKHR S256 * |
| p38MAPK * | phospho-FKHR-FKHRL1 T24-T32 * |
| p70S6K * | phospho-GSK3α-β S21-9 * |
| PI3K ^{†} | phospho-GSK3-α S21 * |
| PKC-α ^{%} | phospho-GSK3β T-Y * |
| PTEN * | phospho-Histone H3 S10 ^{%} |
| SAP-JNK * | phospho-IkB-α S32 * |
| Smac-DIABLO * | phospho-IkB-α S32-36 ^{†} |
| Stat3 * | phospho-IRS-1 S612 * |
| | phospho-LKB1 S334 * |
| | phospho-MARCKs S152 * |
| | phospho-MEK S217-221 * |
| | phospho-mTOR S2448 * |
| | phospho-mTOR S2481 * |
| | phospho-NFkB S536 * |
| | phospho-p38MAPKT180-Y182 * |
| | phospho-p70S6K S371 * |
| | phospho-p70S6K T389 * |
| | phospho-p70S6K T412 ^{%} |
| | phospho-PDGFR Y716 * |
| | phospho-PDGFR Y751 * |
| | phospho-SAP-JNK T183-185 * |
| | phospho-Shc Y317 * |
| | phospho-STAT3 S727 * |
| | phospho-STAT3 Y705 ^{%} |
| | phospho-VEGFR Y1175 * |
| | phospho-VEGFR Y951 * |

Symbol Code ‘Company’
Cell Signaling (Danvers, MA, USA) = *
Upstate Biotechnology (Lake Placid, NY, USA) = ^{%}
Zymed (San Francisco, CA) = ^{&}
DAKO (Carpinteria, CA, USA) = ^{#}
Miltenyi Biotec (Bergisch Gladbach, Germany) = ^{‡}
BD Biosciences (Franklin Lakes, NJ, USA) = ^{†}
Biosource (Camarillo, CA, USA) = **

Total protein values were assessed by staining three slides per set with Sypro Ruby Blot Stain (Molecular Probes, Eugene, Oreg.). Stained slides were scanned on Novaray scanner (Alpha Innotech, San Leandro, Calif., USA) and 16-bit images at 10 μm resolution were generated.

### Statistical Analysis

Spot quantification was performed by using MicroVigene v2.9.9.9 software (Vigenetech, Carlisle, Mass.); secondary antibody staining was subtracted and normalization to total protein was performed on neat spot values. Final normalized signal intensities were incorporated into either SAS v8.01 or JMP v5.1 (Sas Institute Inc., Cary, N.C., USA). In unsupervised analyses hierarchical clustering (Ward method) and Principal Component Analysis (PCA) were performed. Spot intensity values from the chemotherapeutic drugs experiment were further normalized to the correspondent untreated samples at each time point. In supervised analyses hypothesis testing was done by means of non-parametric Wilcoxon rank sum and Kruskal-Wallis tests. A 0.05% false discovery rate was accepted as a cut-off value for statistical significance.

### Results

All the CSCs used in this study have been derived from tumor specimens subjected to mechanical and/or enzymatic dissociation and were subsequently cultured into a serum-free medium supplemented with EGF and bFGF [10,11]. In order to elucidate the nature of signaling networks active in CSCs in the presence or in the absence of growth factors, we starved 15 glioblastoma and 4 colon cancer stem cell lines from growth factors (GFs) for 6 days (average time required to stop cell growth after GFs withdrawal). We then performed RPPMAs (reverse phase protein microarray analysis) with such samples and used normalized intensities of the 98 endpoints evaluated to carry out supervised and unsupervised analyses.

Table 2 shows the proteins that were statistically different by Wilcoxon rank-sum test as performed between CSCs cultured in the presence or in the absence of growth factors for six days.

TABLE 2

| | |
|---|---|
| Endpoint | P value |

Glioblastoma

| | |
|---|---|
| 4EBP1 | 0.006328502 |
| Bcl-X_{L} | 0.044323239 |
| Caspase 3 | 0.036912546 |
| Caspase 8 | 0.026972564 |
| c-Kit | 0.033996266 |
| Cleaved Caspase3 D175 | 0.027565188 |
| Cyclin D1 | 0.018783505 |
| EGF-R | 8.05283E−05 |
| ErbB3 | 0.054653319 |
| GSK3β | 0.047879504 |
| HIF1α | 0.00344502 |
| MEK | 0.002366314 |
| p27/Kip1 | 0.000708897 |
| phospho-4EBP1 S65 | 0.030924258 |
| phospho-4EBP1 T70 | 0.015806976 |
| phospho-Bad S112 | 0.013346183 |
| phospho-Bcl2 S70 | 0.039842087 |
| phospho-cKit Y703 | 0.045961419 |
| phospho-cRaf S338 | 0.037355762 |
| phospho-elF4G S1108 | 0.006411435 |
| phospho-ErbB3 Y1289 | 0.054653319 |
| phospho-ERK T202/Y204 | 0.003064616 |
| phospho-Estrogen Receptor-α S118 | 0.005329739 |
| phospho-FADD S194 | 0.002597857 |
| phospho-FAK Y397 | 0.000834689 |
| phospho-GSK3α S21 | 0.017228256 |
| phospho-IkBα S32/36 | 0.016479547 |
| phospho-NF-kB S536 | 0.043333143 |
| phospho-SAP/JNK T183/185 | 0.001943205 |

Colon

| | |
|---|---|
| EGF-R | 0.031324131 |
| phospho-c-abl T735 | 0.027486336 |
| phospho-c-abl Y345 | 0.014305878 |
| phospho-ERK T202/Y204 | 0.018129008 |

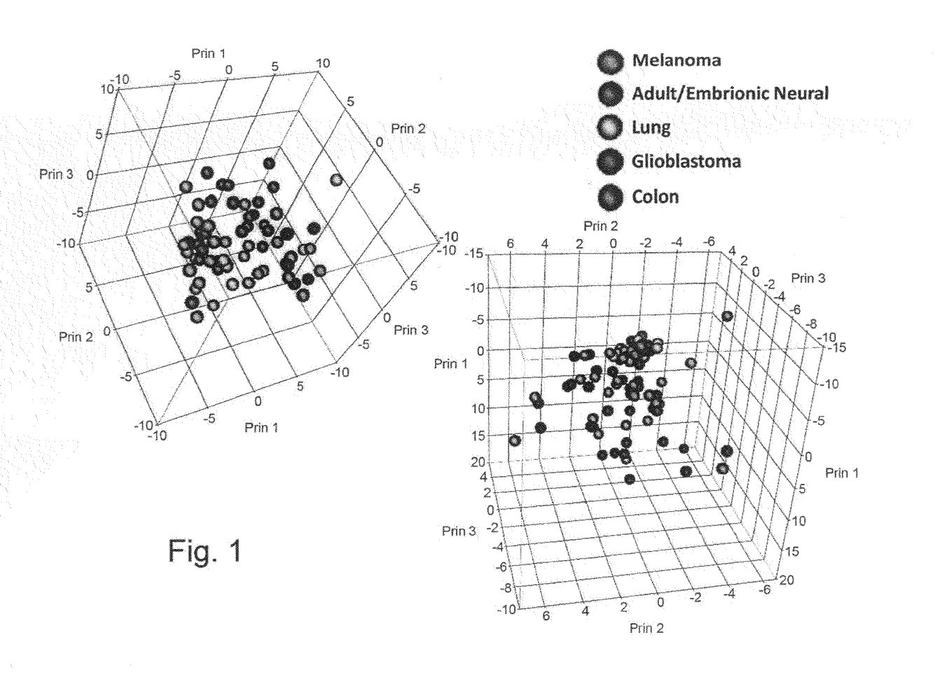

In order to better understand the signaling networks that underlie CSCs growth and survival, we performed principal component analysis (PCA, FIG. 1), a statistical method to reduce the dimensionality of the dataset. We found that CSCs cluster in a tumor-specific manner, regardless of the presence or absence of growth factors.

### Cancer Stem Cell Identifiers

However, within each tumor type, diversity can be noticed in the positioning of the various cell lines inside the three-dimensional space of the first three factors or principal components (PCs). Lung cancer stem cell lines are a clear example of the heterogeneity inside each cancer stem cell population, as two of them clustered close to melanoma CSCs while two others co-clustered with glioblastoma cell lines. Nevertheless, the percentage of variation explained by the first PCs was only 34% on average, meaning that not all of the information contained inside the dataset was uncovered by PCA analysis.

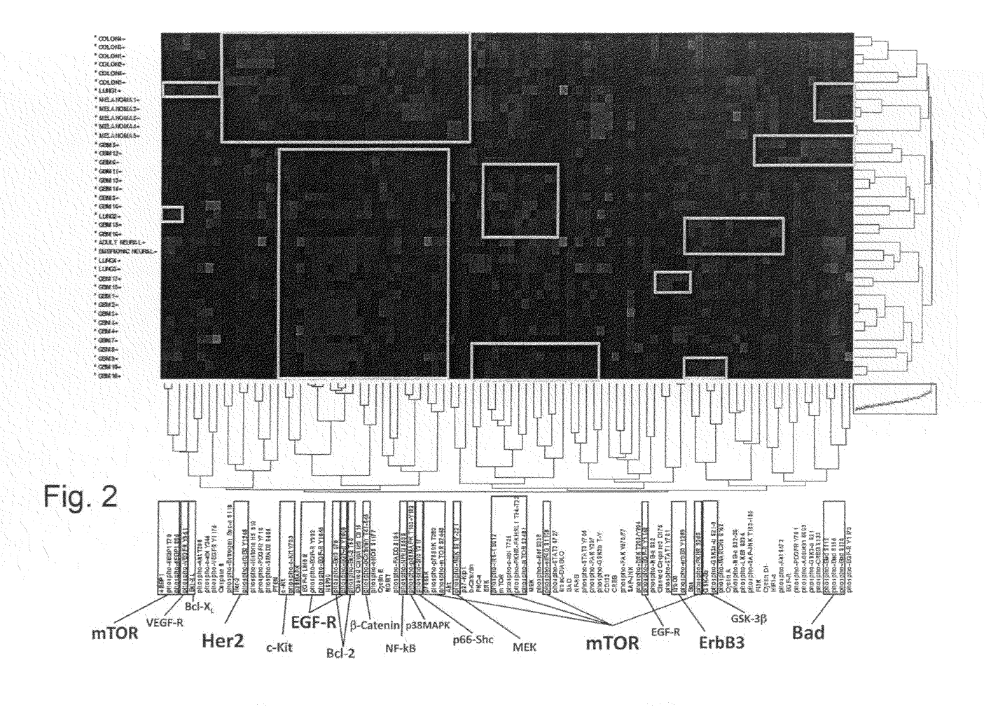

We then decided to confirm the results obtained by doing hierarchical clustering (HCL) only on cancer stem cell lines cultured in the presence of growth factors. HCL (FIG. 2A) revealed that all the CSCs examined share EGF-R activation and Bcl-2 hyper-phosphorylation, but each different subset has its own characteristics.

Melanoma stem cells display enhanced p38MAPK, NF-κB and Shc phosphorylation, colon carcinoma stem cells have hyper-activated HER2 signaling and lung CSCs clustered in three subgroups.

Two out of three lung CSCs show mTOR pathway hyper-activation while one lung CSC has hyper-phosphorylated Bad levels. Glioblastoma stem cell lines cluster into two major groups, characterized by similarity to adult or embryonic neural stem cells. In general, the first group had high levels of both EGF-R and mTOR signaling, while the second group showed down-modulated EGF-R activity, mTOR pathway and GSK3β activation, with two cell lines displaying hyper-phosphorylation of ErbB3.

Notably, one melanoma and one glioblastoma cell line co-clustered because of high levels of phosphorylated Bad and Adducin. In order to confirm such differences from a statistical point, we compared the characteristics of each CSC tumor-type in respect to the others. Table 3 shows the endpoints that are significantly different when CSCs belonging to a class of tumor are compared to all other tumor-types, at basal culturing conditions (presence of growth factors).

TABLE 3

| | |
|---|---|
| Endpoint | P value |

Glioblastoma

| | | |
|---|---|---|
| 4EBP1 | 0.000280477 | ↓ |
| BAD | 0.025065776 | ↑ |
| Bax | 0.015181368 | ↑ |
| c-Kit | 0.0057127 | ↓ |
| Cleaved Caspase 3 D175 | 0.007076884 | ↑ |
| Cleaved Caspase 9 D315 | 0.030510973 | ↓ |
| Cyclin A | 0.045043796 | ↑ |
| EGF-R | 0.050261123 | ↓ |
| EGF-R L858R | 0.000769103 | ↓ |
| ERK | 0.002643889 | ↑ |
| HSP90 | 0.007686052 | ↓ |
| p38MAPK | 0.008113047 | ↓ |
| phospho-4EBP1 T70 | 0.008876469 | ↓ |
| phospho-Akt S473 | 6.40695E−06 | ↑ |
| phospho-Bad S112 | 0.00358245 | ↑ |
| phospho-cRaf S338 | 0.003404947 | ↑ |
| phospho-EGF-R Y1045 | 0.00171626 | ↓ |
| phospho-EGF-R Y1068 | 0.018611535 | ↓ |
| phospho-EGF-R Y992 | 0.007443332 | ↓ |
| phospho-ERK T202/Y204 | 0.001559783 | ↑ |
| phospho-FAK Y397 | 0.000952924 | ↑ |
| phospho-FKHR/FKHRL1 | 0.000508738 | ↑ |
| T24-32 | | |
| phospho-FKHR_S256 | 0.000297749 | ↑ |
| phospho-GSK3α S21 | 0.024105448 | ↑ |
| phospho-GSK3α/β S21-9 | 0.02835041 | ↑ |
| phospho-LKB1 S334 | 0.008908808 | ↑ |
| phospho-MARCKs_S152 | 9.05762E−05 | ↑ |
| phospho-NFkB S536 | 0.025270575 | ↓ |
| phospho-p38MAPK | 0.004365576 | ↓ |
| T180/Y182 | | |
| phospho-PDGFR Y751 | 0.01246587 | ↓ |
| phospho-SAP/JNK T183- | 0.03320161 | ↑ |
| 185 | | |
| phospho-Shc Y317 | 0.03498946 | ↓ |
| phospho-STAT1 Y701 | 0.004360936 | ↑ |
| phospho-STAT3 Y705 | 0.033159897 | ↑ |
| phospho-VEGFR Y951 | 0.012842423 | ↓ |
| PI3K | 0.000377322 | ↑ |
| PKCα | 1.19626E−05 | ↑ |

Melanoma

| | | |
|---|---|---|
| 4EBP1 | 0.0356919 | ↑ |
| BAD | 0.000315527 | ↓ |
| Bcl-X_{L} | 0.028800402 | ↓ |
| CD133 | 0.00096916 | ↓ |
| Cyclin E | 0.005557025 | ↑ |
| EGF-R L858R | 0.045852227 | ↑ |
| ERK | 0.039550627 | ↓ |
| HIF1α | 0.000488379 | ↓ |
| p38MAPK | 0.008624549 | ↑ |
| p70S6K | 0.001215839 | ↑ |
| phospho-βCatenin | 0.039550627 | ↓ |
| T41/S45 | | |
| phospho-c-abl T735 | 0.005557025 | ↓ |
| phospho-EGF-R Y1045 | 0.014774497 | ↑ |
| phospho-EGF-R Y992 | 0.0190451 | ↑ |
| phospho-Estrogen | 0.005183578 | ↓ |
| Receptor-α S118 | | |
| phospho-FAK Y397 | 0.000355764 | ↓ |
| phospho-FKHR/FKHRL1 | 0.002164987 | ↓ |
| T24-32 | | |
| phospho-GSK3α Y279/β | 0.000302407 | ↓ |
| Y216 | | |
| phospho-Histone-H3 S10 | 0.019716355 | ↓ |
| phospho-lkBα S32-36 | 0.002164987 | ↓ |
| phospho-LKB1 S334 | 0.002661393 | ↓ |
| phospho-MEK S217-221 | 0.004871383 | ↑ |
| phospho-NFkB S536 | 0.008216221 | ↑ |
| phospho-p38MAPK | 0.001047979 | ↑ |
| T180/Y182 | | |
| phospho-p70S6K T389 | 0.005208599 | ↑ |
| phospho-Shc Y317 | 0.050857738 | ↑ |
| phospho-STAT1 Y701 | 0.001306177 | ↓ |
| PI3K | 0.005918788 | ↓ |

Colon

| | | |
|---|---|---|
| 4EBP1 | 0.045589156 | ↑ |
| CD133 | 0.026294451 | ↑ |
| Cyclin E | 0.026312267 | ↓ |
| ErbB3 | 0.029479735 | ↑ |
| Her2 | 0.004465258 | ↑ |
| p70S6K | 0.043202179 | ↓ |
| phospho-Adducin | 0.050612432 | ↓ |
| S663 | | |
| phospho-Akt S473 | 0.01448386 | ↓ |
| phospho-Bad S112 | 0.034815908 | ↓ |
| phospho-cRaf S338 | 0.001008585 | ↓ |
| phospho-eIF4G | 0.00187194 | ↓ |
| S1108 | | |
| phospho-ErbB2 | 0.007968606 | ↑ |
| Y1248 | | |
| phospho-erbB3 | 0.029479735 | ↑ |
| Y1289 | | |
| phospho-ERK | 0.001869605 | ↓ |
| T202/Y204 | | |
| phospho-FKHR S256 | 0.000448317 | ↓ |
| phospho-GSK3α S21 | 0.00387014 | ↓ |
| phospho-GSK3α/β | 0.012840586 | ↓ |
| S21-9 | | |
| phospho-HistoneH3 | 0.003611295 | ↑ |
| S10 | | |
| phospho-lkBα S32 | 0.045589156 | ↓ |
| phospho-MARCKs | 0.00862359 | ↓ |
| S152 | | |
| phospho-MEK | 0.010615413 | ↓ |
| S217-221 | | |
| phospho-PDGFR | 0.003366491 | ↑ |
| Y716 | | |
| phospho-PDGFR | 0.005433461 | ↑ |
| Y751 | | |
| phospho-SAP/JNK | 0.005125886 | ↓ |
| T183-185 | | |
| phospho-VEGFR | 0.002920168 | ↑ |
| Y951 | | |
| PI3K | 0.02944123 | ↓ |

Lung

| | | |
|---|---|---|
| CD133 | 0.002608702 | ↑ |
| ERK | 0.041669665 | ↓ |
| GSK3β | 0.037213726 | ↓ |
| phospho-Adducin | 0.012429351 | ↓ |
| S663 | | |
| phospho-Akt S473 | 0.026201899 | ↓ |
| phospho-GSK3α/β | 0.023299427 | ↓ |
| S21-9 | | |
| phospho-mTOR | 0.001401205 | ↓ |
| S2481 | | |
| PKCα | 0.000189908 | ↓ |

### Induction of Differentiation

An intriguing anti-cancer strategy is thought to be the induction of differentiation of CSCs in order to deplete the tumor-regeneration cell reservoir. Several papers demonstrated the importance of molecular dissection of cancer cells aimed to differentiative targeted therapy [29,30,31]. Likewise, we were interested in identifying proteins differentially regulated before and after induction of differentiation of CSCs.

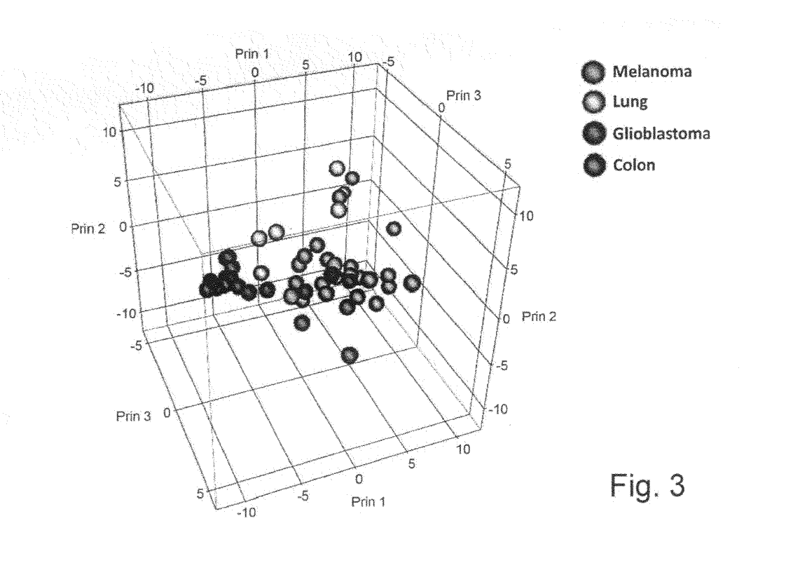

18 glioblastoma, 1 colon, 4 lung and 3 melanoma stem cell samples were subjected to differentiation experiment and then printed for RPPMAs. PCA over 63 endpoints shows that CSCs have tumor-specific response, maintaining their own niche in the 3D space of the first three PCs. Moreover, the extent to which differentiation induction changes the signaling inside CSCs is clearly visible (FIG. 3).

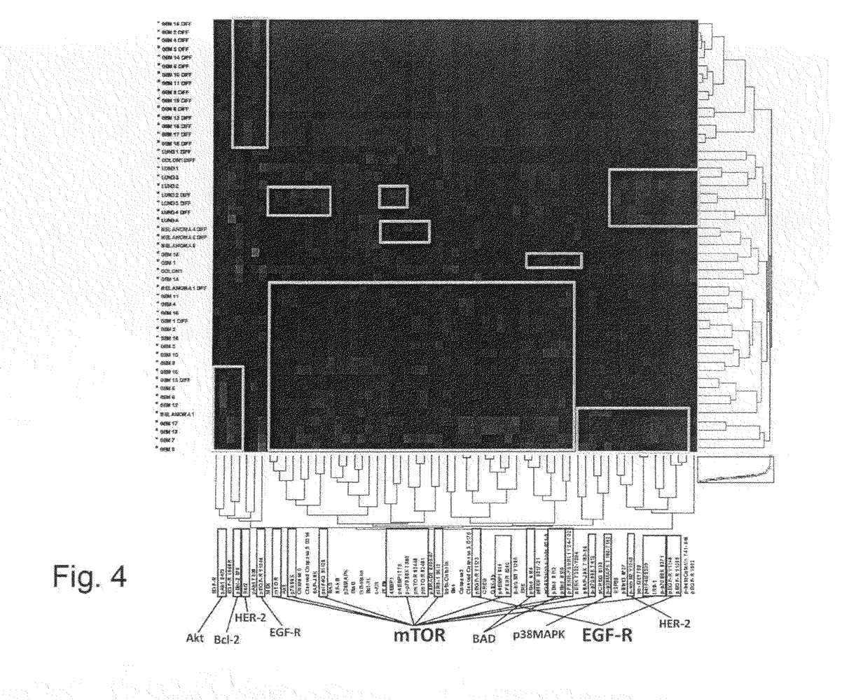

Similarly to starvation experiments, the amount of information that we were able to extract from the data through PCA alone was limited (cumulative percentage of variation explained by three PCs was 60%). However, by HCL analysis we observed that differentiation induction in CSCs caused a general down-modulation of the majority of the endpoints tested (FIG. 4).

As predicted by PCA analysis, the results show a clear separation between differentiated and undifferentiated CSCs, which is particularly significant for glioblastoma stem cells. A tumor-wise clustering is also evident and, interestingly, the central part of the heat-map shows a partially overlapping signaling for differentiated and undifferentiated melanoma and lung CSCs.

Upon differentiation, glioblastoma stem cells lose mTOR pathway activation and maintain discrete levels of phospho-EGF-R Y1045 and phospho-Bcl-2 S70. After differentiation induction melanoma stem cells display high levels of 4EBP1, p70S6K and mTOR phosphorylation, while three out of four lung-derived CSCs show high pospho-eEF4E S209 levels. Although only one colon carcinoma CSC cell line was included in the differentiation experiments, after its differentiation we observed an up-regulation of phospho-Bcl-2 S70. Supervised analysis partially confirmed the results obtained by HCL (Table 4, which shows the in CSCs after differentiation induction as compared to CSCs cultured in the presence of growth factors).

TABLE 4

| | |
|---|---|
| Endpoint | P value |

Glioblastoma

| | | |
|---|---|---|
| 4EBP1 | 8.05987E−07 | ↓ |
| Akt | 3.45112E−06 | ↓ |
| BAD | 5.12704E−07 | ↓ |
| Bax | 3.97338E−06 | ↓ |
| Bcl-X_{L} | 4.57092E−06 | ↓ |
| bActin | 1.25768E−06 | ↓ |
| bCatenin | 6.92466E−06 | ↓ |
| c-Kit | 2.98184E−05 | ↓ |
| Caspase 3 | 5.12704E−07 | ↓ |
| Caspase 9 | 9.35619E−07 | ↓ |
| Cleaved Caspase 3 D175 | 3.77661E−07 | ↓ |
| Cleaved Caspase 9 D315 | 0.000147455 | ↓ |
| CREB | 1.94802E−06 | ↓ |
| EGF-R | 0.011087054 | ↓ |
| EGF-R L858R | 0.001076191 | ↓ |
| ERK | 1.25768E−06 | ↓ |
| GSK3β | 5.12704E−07 | ↓ |
| Her2 | 0.009367774 | ↓ |
| HSP90 | 1.25768E−06 | ↓ |
| IkB-α | 2.99504E−06 | ↓ |
| IRS-1 | 6.40836E−06 | ↓ |
| MEK | 1.47981E−07 | ↓ |
| mTOR | 1.68503E−06 | ↓ |
| NF-κB | 8.05987E−07 | ↓ |
| phospho-bCatenin T41/S45 | 9.35619E−07 | ↓ |
| phospho-ErbB2 Y1248 | 2.99504E−06 | ↓ |
| phospho-ErbB3 Y1289 | 9.35619E−07 | ↓ |
| phospho-p38MAPK | 9.09668E−06 | ↓ |
| T180/Y182 | | |
| phospho-p70S6K S371 | 3.97338E−06 | ↓ |
| phospho-p70S6K T389 | 4.948E−05 | ↓ |
| phospho-p70S6K T412 | 8.10443E−05 | ↓ |
| p38MAPK | 5.12704E−07 | ↓ |
| phospho-4EBP1 S65 | 2.25019E−06 | ↓ |
| phospho-4EBP1 T70 | 2.98184E−05 | ↓ |
| p70S6K | 2.77271E−07 | ↓ |
| phospho-Akt S473 | 0.047117464 | ↓ |
| phospho-Akt T308 | 0.018041051 | ↓ |
| phospho-Bad S112 | 6.93743E−07 | ↓ |
| phospho-Bad S136 | 8.05987E−07 | ↓ |
| phospho-Bad S155 | 6.93743E−07 | ↓ |
| phospho-cKit Y703 | 1.04134E−05 | ↓ |
| phospho-CREB S133 | 3.45112E−06 | ↓ |
| phospho-EGF-R Y1045 | 0.000786804 | ↓ |
| phospho-EGF-R Y1068 | 6.92466E−06 | ↓ |
| phospho-EGF-R Y1148 | 5.96639E−07 | ↓ |
| phospho-EGF-RY1173 | 5.96639E−07 | ↓ |
| phospho-eIF4G S1108 | 2.62188E−05 | ↓ |
| phospho-FKHR/FKHRL1 | 2.99504E−06 | ↓ |
| T24-32 | | |
| phospho-FKHR S256 | 1.68503E−06 | ↓ |
| phospho-GSK3a/b S21-9 | 1.55449E−05 | ↓ |
| phospho-IRS1 S612 | 1.77368E−05 | ↓ |
| phospho-MEK S217-21 | 1.25768E−06 | ↓ |
| phospho-mTOR S2448 | 5.25402E−06 | ↓ |
| phospho-mTOR S2481 | 4.57092E−06 | ↓ |
| phospho-SAP/JNK T183-85 | 1.08521E−06 | ↓ |
| phospho-SMAD2 S465-67 | 2.3035E−05 | ↓ |
| phospho-Stat3 S727 | 1.94802E−06 | ↓ |
| PTEN | 0.002173438 | ↓ |
| SAP/JNK | 0.000629624 | ↓ |
| Stat3 | 2.25019E−06 | ↓ |

Melanoma

| | | |
|---|---|---|
| Akt | 0.04953461 | ↑ |
| c-Kit | 0.04953461 | ↓ |
| CREB | 0.04953461 | ↓ |
| IRS-1 | 0.04953461 | ↓ |
| phospho-p70S6K S371 | 0.04953461 | ↓ |
| phospho-Akt S473 | 0.04953461 | ↑ |
| phospho-Akt T308 | 0.04953461 | ↑ |
| phospho-Bad S136 | 0.04953461 | ↓ |
| phospho-ERK T202/Y204 | 0.04953461 | ↑ |
| phospho-FKHR/FKHRL1 | 0.04953461 | ↓ |
| T24-T32 | | |
| phospho-FKHR S256 | 0.04953461 | ↑ |
| phospho-IRS-1 S612 | 0.04953461 | ↑ |
| phospho-mTOR S2481 | 0.04953461 | ↑ |
| PTEN | 0.04953461 | ↓ |

Lung

| | | |
|---|---|---|
| Cleaved Caspase 9 D315 | 0.020921335 | ↓ |
| Akt | 0.043308143 | ↓ |
| phospho-4EBP1 T70 | 0.043308143 | ↓ |

### Figure Legends for Example 1

FIG. 1. Score plots of Principal Component Analyses (PCA) on starvation experiment. PCA was performed on a total of 98 endpoints measured over 15 glioblastoma and 4 colon cancer stem cell lines cultured in the presence (dark gray) and in the absence (light gray) of growth factors. 4 lung and 5 melanoma CSCs were also included in this analysis, but they were only cultured in complete medium and were not subjected to starvation. The two graphs show score plots from two replicate experiments where the second one was performed with 70% of the total number of endpoints valuated in the study and contains biological replicates for glioblastoma stem cells.

FIG. 2. Hierarchical Clustering (HCL) of CSCs cultured in the presence of growth factors. Ward method was used to construct HCL trees. Cluster distance graph is shown at the right bottom of the heat-map. The clusters formed by different CSCs are highlighted by colors and by yellow boxes while at the bottom are the names of the pathways to which the selected endpoints belong.

FIG. 3. Score plots of Principal Component Analyses (PCA) on differentiation experiment. PCA was performed on a total of 63 endpoints measured over 17 glioblastoma, 1 colon, 3 melanoma an 4 lung cancer stem cell lines after induction of differentiation (dark colored) and in complete medium (light colored).

FIG. 4. Hierarchical Clustering of CSCs before and after induction of differentiative program. Ward method was used to construct HCL trees. Cluster distance graph is shown at the right bottom of the heat-map. The clusters formed by different CSCs are highlighted by colors and by yellow boxes while at the bottom are the names of the pathways to which the selected endpoints belong.

### REFERENCES

All references cited herein are herby incorporated by reference, unless otherwise apparent.
1 WICHA MS, LIU S, DONTU G: Cancer stem cells: an old idea—a paradigm shift. Cancer research (2006) 66(4):1883-1890; discussion 1895-1886.
2 COHNHEIM V. Congenitales, quergestreiftes muskelsarkom der nieren. In: Virchows Arch. Pathol. Anat. Physiol. Klin. Med;. 1875:64-69.
3 POLYAK K, HAHN WC: Roots and stems: stem cells in cancer. Nature medicine (2006) 12(3):296-300.
4 MCCLANAHAN T, KOSEOGLU S, SMITH K et al.: Identification of overexpression of orphan G protein-coupled receptor GPR49 in human colon and ovarian primary tumors. Cancer biology & therapy (2006) 5(4):419-426.
5 BARKER N, VAN ES JH, KUIPERS J et al.: Identification of stem cells in small intestine and colon by marker gene Lgr5. Nature (2007) 449(7165):1003-1007.
6 BONNET D, click JE: Human acute myeloid leukemia is organized as a hierarchy that originates from a primitive hematopoietic cell. Nature medicine (1997) 3(7):730-737.
7 REYA T, MORRISON S J, CLARKE M F, WEISSMAN I L: Stem cells, cancer, and cancer stem cells. Nature (2001) 414(6859):105-111.
8 SINGH SK, HAWKINS C, CLARKE ID et al.: Identification of human brain tumour initiating cells. Nature (2004) 432(7015):396-401.
9 O'BRIEN CA, POLLETT A, GALLINGER S, Dick J E: A human colon cancer cell capable of initiating tumour growth in immunodeficient mice. Nature (2007) 445(7123):106-110.
10 RICCI-VITIANI L, LOMBARDI D G, PILOZZI E et al.: Identification and expansion of human colon-cancer-initiating cells. Nature (2007) 445(7123):111-115.
11 ERAMO A, LOTTI F, SETTE G et al.: Identification and expansion of the tumorigenic lung cancer stem cell population. Cell Death Differ (2007)
12 COLLINS A T, BERRY P A, HYDE C, STOWER M J, MAITLAND N J: Prospective identification of tumorigenic prostate cancer stem cells. Cancer research (2005) 65(23): 10946-10951.
13 AL-HAJJ M, WICHA M S, BENITO-HERNANDEZ A, MORRISON S J, CLARKE M F: Prospective identification of tumorigenic breast cancer cells. Proceedings of the National Academy of Sciences of the United States of America (2003) 100(7):3983-3988.
14 LI C, HEIDT D G, DALERBA P et al.: Identification of pancreatic cancer stem cells. Cancer research (2007) 67(3):1030-1037.
15 SCHATTON T, MURPHY G F, FRANK NY et al.: Identification of cells initiating human melanomas. Nature (2008) 451(7176):345-349.
16 MICHOR F, HUGHES T P, IWASA Y et al.: Dynamics of chronic myeloid leukaemia. Nature (2005) 435(7046): 1267-1270.
17 TANG C, ANG B T, PERVAIZ S: Cancer stem cell: target for anti-cancer therapy. Faseb J (2007) 21(14):3777-3785.
18 DEAN M: Cancer stem cells: redefining the paradigm of cancer treatment strategies. Molecular interventions (2006) 6(3): 140-148.
19 KITANO H: Cancer as a robust system: implications for anticancer therapy. Nature reviews (2004) 4(3):227-235.
20 MAHTANI R L, MACDONALD J S: Synergy between cetuximab and chemotherapy in tumors of the gastrointestinal tract. The oncologist (2008) 13(1):39-50.
21 ZHANG H, BEREZOV A, WANG Q et al.: ErbB receptors: from oncogenes to targeted cancer therapies. The Journal of clinical investigation (2007) 117(8):2051-2058.
22 BRADEEN H A, EIDE C A, O'HARE T et al.: Comparison of imatinib mesylate, dasatinib (BMS-354825), and nilotinib (AMN107) in an N-ethyl-N-nitrosourea (ENU)-based mutagenesis screen: high efficacy of drug combinations. Blood (2006) 108(7):2332-2338.
23 CAMPHAUSEN K, TOFILON PJ: Combining radiation and molecular targeting in cancer therapy. Cancer biology & therapy (2004) 3(3):247-250.
24 HOCHHAUS A: Cytogenetic and molecular mechanisms of resistance to imatinib. Seminars in hematology (2003) 40(2 Suppl 2):69-79.
25 PETRICOIN E F, 3RD, ESPINA V, ARAUJO R P et al.: Phosphoprotein pathway mapping: Akt/mammalian target of rapamycin activation is negatively associated with childhood rhabdomyosarcoma survival. Cancer research (2007) 67(7):3431-3440.
26 PETRICOIN E F, 3RD, BICHSEL V E, CALVERT VS et al.: Mapping molecular networks using proteomics: a vision for patient-tailored combination therapy. J Clin Oncol (2005) 23(15):3614-3621.
27 ESPINA V, MEHTA AI, WINTERS ME et al.: Protein microarrays: molecular profiling technologies for clinical specimens. Proteomics (2003) 3(11):2091-2100.
   28 VANMETER A, SIGNORE M, PIEROBON M et al.: Reverse-phase protein microarrays: application to biomarker discovery and translational medicine. Expert review of molecular diagnostics (2007) 7(5):625-633.
29 NAKANO I, SAIGUSA K, KORNBLUM H I: BMPing off glioma stem cells. Cancer cell (2008) 13(1):3-4.
30 PICCIRILLO S G, REYNOLDS B A, ZANETTI N et al.: Bone morphogenetic proteins inhibit the tumorigenic potential of human brain tumour-initiating cells. Nature (2006) 444(7120):761-765.
31 SCHUGAR R C, ROBBINS P D, DEASY B M: Small molecules in stem cell self-renewal and differentiation. Gene therapy (2008) 15(2): 126-135.
32 KANG M K, KANG S K: Tumorigenesis of chemotherapeutic drug-resistant cancer stem-like cells in brain glioma. Stem cells and development (2007) 16(5):837-847.
33 SMITH V, HOBBS S, COURT W et al.: ErbB2 overexpression in an ovarian cancer cell line confers sensitivity to the HSP90 inhibitor geldanamycin. Anticancer research (2002) 22(4):1993-1999.
34 SAWAI A, CHANDARLAPATY S, GREULICH H et al.: Inhibition of Hsp90 down-regulates mutant epidermal growth factor receptor (EGFR) expression and sensitizes EGFR mutant tumors to paclitaxel. Cancer research (2008) 68(2):589-596.

### Example 2

### RPPMA-Guided Pathway Analysis in Colon Cancer Stem Cells

Colorectal cancer is the third most common type of non-skin cancer in men (after prostate cancer and lung cancer) and in women (after breast cancer and lung cancer). It is the second leading cause of cancer death in the United States after lung cancer [1, see “References for Example 2,” below]. Conventional chemotherapy for colorectal cancer patients has been potentiated by novel therapeutic agents such as Cetuximab or Bevacizumab. Although promising, there are still many issues that need to be addressed regarding these agents, like the accurate identification of target patients [2]. The identification and in vitro expansion of colon cancer initiating cells has added a fundamental concept to colorectal cancer pathophysiology, where cancer stem cells are putatively responsible for relapses and resistance to treatments [3,4].

Colon cancer stem cells (CSCs) represent a valuable tool for in vitro drug screening and with the aid of pathway analysis platforms such as reverse phase microarrays (RPPMA), active compounds might be discovered that more closely represent an effective in vivo therapy. Reverse phase microarray analysis allowed us to choose a set of inhibitors that, based on specific protein kinase hyperactivity, would target colon CSCs. A preliminary in vitro experiment showed that most kinase inhibitors are not effective on colon cancer stem cells while effects on viability are evident after 60 hours of incubation with HSP90 inhibitor geldanamycin.

Geldanamycin was first isolated as the fermentation product of Streptomyces hygroscopicus. This general class of benzoquinone ansamycins first became of interest in the 1980s as potential tyrosine kinase inhibitors and their mechanism of action has been elucidated. Geldanamycin inhibits HSP90 chaperon function, therefore blocking the function of many client proteins that are involved in cancer survival/progression [5]. Since bioavailable analogs of geldanamycin have been developed that are already in clinical trials [6,7], we decided to find partner compounds for geldanamycin in order to build effective combination therapies for colorectal cancer. Thus, we performed pathway hunting on colon CSCs by using a commercially available kinase inhibitor library.

Moreover, since it has already been shown that 17-AAG (17-Allylamino-17-demethoxygeldanamycin) sensitizes colon cancer cell lines (normal cell lines, not colon cancer stem cell lines) to TRAIL (TNF-related Apoptosis-Inducing Ligand) [8], we treated colon cancer stem cells with geldanamycin analogs and TRAIL as single agents and in combination with 17-AAG or 17-DMAG (17-dimethylaminoethylamino-17-demethoxy-geldanamycin).

These experiments were conducted in the presence of at least one growth factor (namely EGF and bFGF), and the culturing conditions were identical to those utilized for preparing samples for RPPMA analysis in Example 1, so as to maintain the colon cancer stem cells in culture.

### Legend to Figures for Example 2

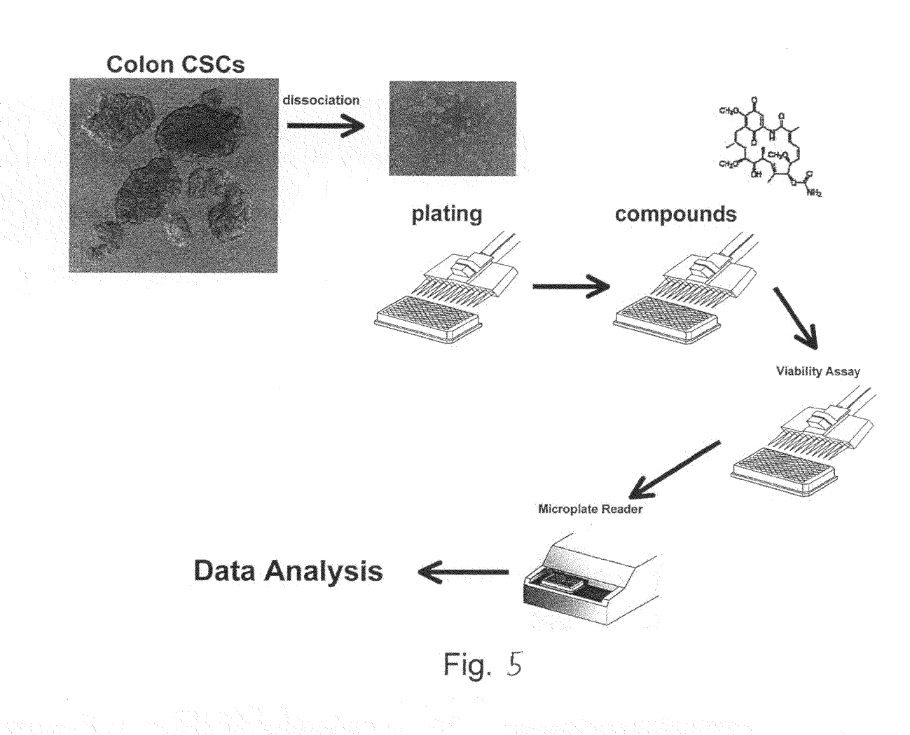

FIG. 5 shows drug screening experimental settings.

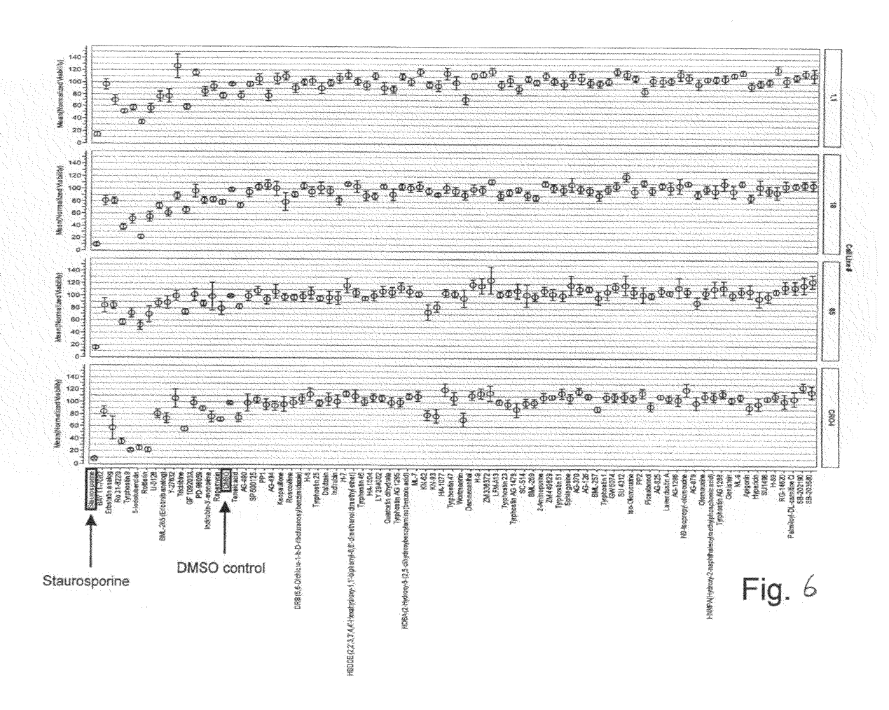

FIG. 6 shows means and 95% confidence intervals of normalized results obtained from three independent experiments on four different colon CSC lines are plotted against kinase inhibitors.

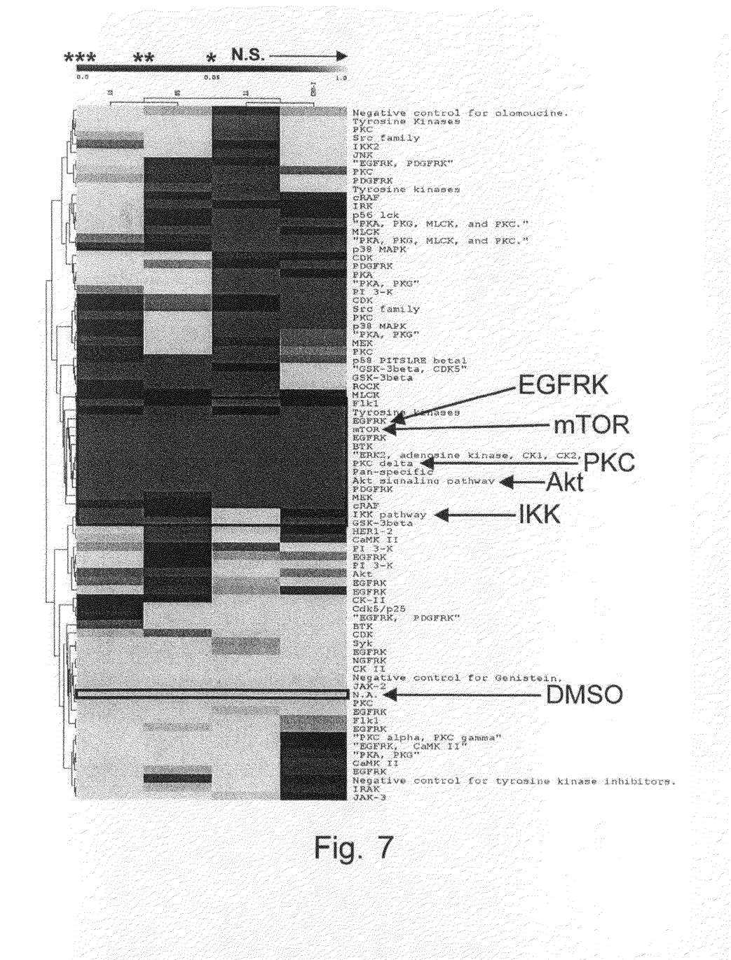

FIG. 7 shows hierarchical clustering of statistical significance (p values) for each kinase inhibitor over the colon CSCs. Inhibitors are labeled on the right with the name of the targeted protein kinase activity. N.S. states for non-significant.

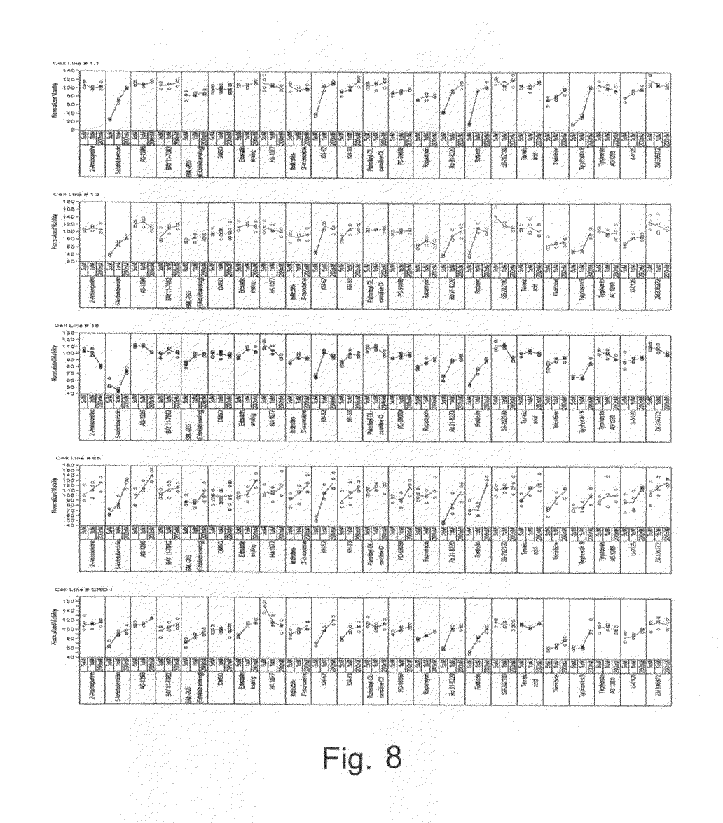

FIG. 8 is a point chart of the results from the titration experiment on various colon CSCs. Normalized viability triplicates are plotted against inhibitors within three different concentration levels.

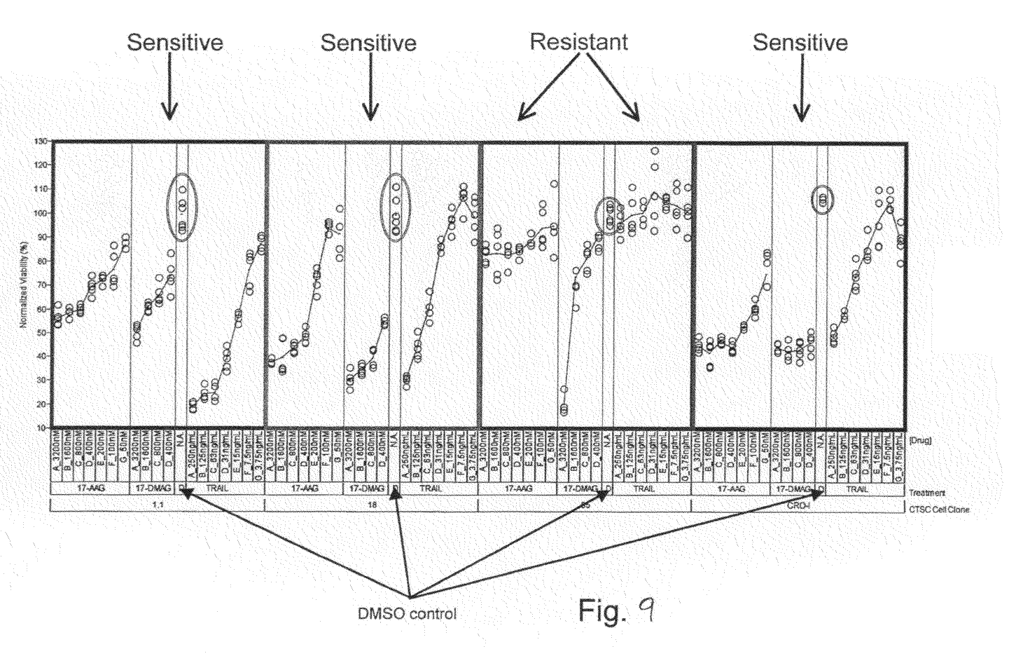

FIG. 9 is a point chart of the results from dose-response analysis on four colon CSCs. Normalized viability (5 replicates) is plotted against escalating doses of TRAIL, 17-AAG and 17-DMAG.

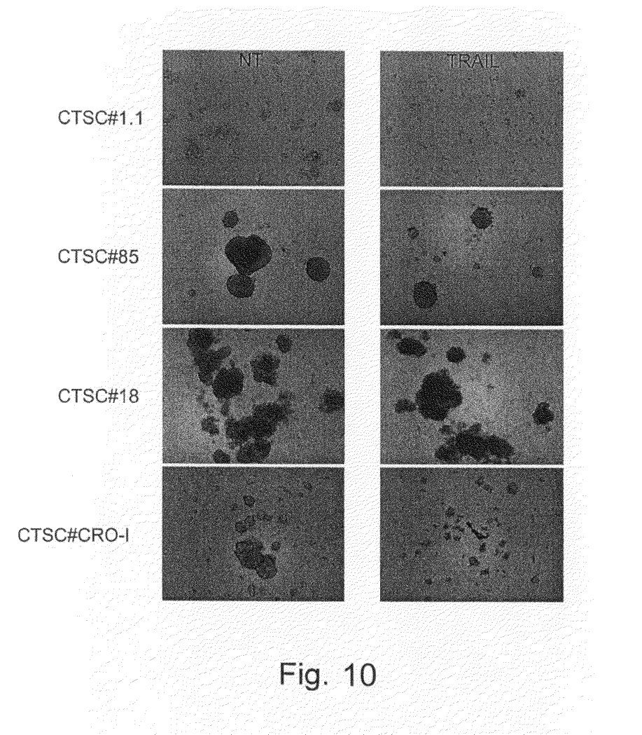

FIG. 10 shows 20× Phase contrast images of colon CSCs untreated or cultivated 48 h in the presence of 62 ng/mL TRAIL.

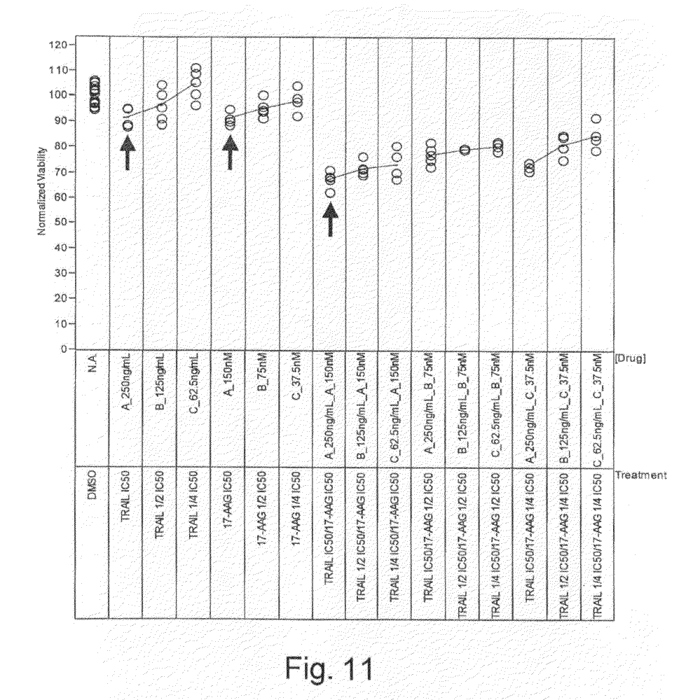

FIG. 11 is a point chart of the drug combination experiment on CTSC#85 (TRAIL-resistant colon CSC line). Normalized viability at 24 h is plotted against drug combinations. The synergistic effect is highlighted by the centermost (red) arrow for “TRAIL IC50/17-AAG IC50.”

The kinase inhibitor library we have used comprises 80 known kinase inhibitors of well-defined activity. Table 5 contains a detailed list of the inhibitors with their chemical descriptors. A flowchart showing the steps of the screenings performed on colon CSCs, is depicted in FIG. 5. Before treatment with inhibitors, colon CSCs undergo trypsin/EDTA dissociation in order to be consistently plated into 96-well microtiter plates. After 48 h cells recover from dissociation and form spheroids again. Inhibitors are subsequently added to the cells as single agents while ATP-based-viability assay are performed after another 48 h.

FIG. 6 shows the results obtained from three independent experiments by using the kinase inhibitor library. Viability controls are cells treated with DMSO whereas staurosporin-treated cells are positive controls of death. It is evident, in this data representation, that many inhibitors do not affect colon CSCs viability whereas a few lead to either reduction or increase in proliferation.

FIG. 7 depicts the hierarchical clustering of statistical significance for the difference of each inhibitor as compared to DMSO controls. Colon CSCs display specific patterns of sensitivity to kinase inhibitors while having common sensitivity nodes.

This data confirms the RPPMA results. What is surprising is that some inhibitors lead to significant proliferation revealing that cancer cells have complex circuitry where some pathways can be unblocked by microenvironmental clues leading to cell proliferation. Such pro-survival compounds are inhibitors of p38MAPK, c-Raf or PKA/ROCK (NCBI gene IDs are 1432, 5894 and 5566/6093 respectively). On the other hand, drugs that significantly lead to a reduction in colon CSC number are inhibitors of PKC, p70S6K, Akt and MEK1 (NCBI gene IDs are 5578, 6198, 207 and 5604 respectively).

These significantly active compounds have been subsequently titrated down from 5 μM to 200 nM concentration in order to determine unspecificity or toxicity effects. FIG. 8 represents one preliminary titration experiment over five colon CSC lines. Upon dilutions most of the inhibitors show reduction of their activity thus confirming that colon CSC are not oncogene addicted.

Combination of effective compounds with geldanamycin is therefore expected to provide a cocktail of effective drugs for colon cancer therapy. RPPMA-guided pathway analysis is useful, therefore, for individualized patient treatment.

Tumor necrosis factor-related apoptosis-inducing ligand (TRAIL, also known as Apo2L) is a potent inducer of cell death, active mostly against cancer but not normal cells, thus being a perfect candidate for cancer treatment [9]. It has been demonstrated that TRAIL resistance can be overcome by co-treatment with 17-AAG in various tumor cell line models, including colon and lung [7,8]. Nonetheless, no clinical trials are currently investigating a combination treatment of TRAIL and geldanamycin analogs.

We treated colon CSCs with escalating doses of TRAIL and 17-AAG/17-DMAG (FIG. 9) and we calculated IC50 (inhibitory concentration 50) at 36 h (Table 7). One out of four colon CSCs is resistant to TRAIL treatment alone (FIGS. 9-10) while being sensitive to 17-DMAG as single agent. Drug combination experiments demonstrated that the resistant clone is rapidly (24 h) sensitized to TRAIL treatment by 17-AAG (FIG. 11).

Table 6 contains information on inhibitors that were effective on colon CSCs, as from Calbiochem (http://www.merckbiosciences.co.uk/g.asp?f=CBC/home.html) and the DTP Repository (http://www.dtp.nci.nih.gov/docs/dtp-search.html).

### Materials and Methods

For drug screening tests colon CSCs were subjected to enzymatic dissociation with a 0.125% Trypsin/0.5 mM EDTA solution (Invitrogen, San Diego, Calif., USA). Cells were incubated at 37° C. for a variable time, depending on the size and on the sensitivity of each colon CSC line, until single cell suspension was achieved. Cells were then washed once in fresh medium and plated into 96-well microtiter plates in 80 μL volume. The number of cells plated ranged from 2000 to 5000 per well. Drugs were added after 48 h by dispensing each well with 20 μL of medium containing 5× concentrated compounds. We purchased the kinase inhibitor library from Enzo Life Sciences International, Inc (formerly BIOMOL International, L.P., PA, USA) and used each single inhibitor at a final 5RM concentration. As a control DMSO was added to cells at a final concentration of 0.1% (v/v).

Recombinant human Killer TRAIL was purchased from Alexis (San Diego, Calif., USA) while Geldanamycin, 17-AAG and 17-DMAG were purchased from Calbiochem (Nottingham, UK). All agents were diluted into fresh medium at the specified concentrations.

Measure of cell viability was performed using CelltiterGlo® luminescent assay (Promega Corporation, Madison, Wis., USA) and luminescence was recorded on Beckman Coulter DTX880 multimode reader (Beckman Coulter Inc., Fullerton, Calif., USA) with an integration time of 100 ms.

Statistical analysis and IC50 calculations were performed with the aid of Microsoft Excel (Microsoft Corporation, www.microsoft.com) and GraphPad Prism version 4.00 for Windows, (GraphPad Software, San Diego, Calif., www.graphpad.com) while hierarchical clustering was performed with TMev v4.3.01 [10]. Normalized viability represents the signal intensity of each replicate divided by the average of single plate DMSO controls (in percent). Dunnett's post-test was performed on at least three independent experiments for each colon CSC line to calculate statistical significance versus DMSO controls. P values were subsequently clustered by using Euclidean distance as metric and complete linkage as a linkage method.

### References for Example 2

1 http://www.cancer.org/downloads/STT/2008CAFFfinalsecured.pdf, Cancer Facts and Figures 2008 (2008).
2 IQBAL S, LENZ H J: Integration of novel agents in the treatment of colorectal cancer. Cancer chemotherapy and pharmacology (2004) 54 Suppl 1:S32-39.
3 BOMAN B M, HUANG E: Human colon cancer stem cells: a new paradigm in gastrointestinal oncology. J Clin Oncol (2008) 26(17):2828-2838.
4 RICCI-VITIANI L, LOMBARDI D G, PILOZZI E et al.: Identification and expansion of human colon-cancer-initiating cells. Nature (2007) 445(7123):111-115.
5 ZHANG H, BURROWS F: Targeting multiple signal transduction pathways through inhibition of Hsp90. Journal of molecular medicine (Berlin, Germany) (2004) 82(8):488-499.
6 SOLIT D B, OSMAN I, POLSKY D et al.: Phase II trial of 17-allylamino-17-demethoxygeldanamycin in patients with metastatic melanoma. Clin Cancer Res (2008) 14(24):8302-8307.
7 WANG X, JU W, RENOUARD J et al.: 17-allylamino-17-demethoxygeldanamycin synergistically potentiates tumor necrosis factor-induced lung cancer cell death by blocking the nuclear factor-kappaB pathway. Cancer research (2006) 66(2):1089-1095.
8 VASILEVSKAYA I A, O'DWYER P J: 17-Allylamino-17-demethoxygeldanamycin overcomes TRAIL resistance in colon cancer cell lines. Biochemical pharmacology (2005) 70(4):580-589.
9 ALMASAN A, ASHKENAZI A: Apo2L/TRAIL: apoptosis signaling, biology, and potential for cancer therapy. Cytokine & growth factor reviews (2003) 14(3-4):337-348.
10 SAEED A I, SHAROV V, WHITE J et al.: TM4: a free, open-source system for microarray data management and analysis. BioTechniques (2003) 34(2):374-378.

### Example 3

### Treatment of Luciferase-Engineered Colon CSCs with TRAIL and/or 17-DMAG

In vivo experiments are performed where luciferase-engineered colon CSCs are injected subcutaneously into NOD/SCID (Non obese diabetic severe combined immunodeficiency) mice. After the growth of the xenograft, mice are treated with both TRAIL and 17-DMAG, alone or in combination.

TABLE 5

| | | | | | | | | PubChem |
|---|---|---|---|---|---|---|---|---|
| PLATE | | | COMPOUND NAME OR ID | | | CONC. | | Info |
| LOCATION | CATALOG # | CAS # | NUMBER | M.W. | SOLVENT | (mM) | TARGET | available? |
| B1 | EI-360 | 167869- | PD-98059 | 267.3 | DMSO | 10 | MEK | Y |
| | | 21-8 | | | | | | |
| B2 | EI-282 | 109511- | U-0126 | 380.5 | DMSO | 10 | MEK | Y |
| | | 58-2 | | | | | | |
| B3 | EI-286 | 152121- | SB-203580 | 377.4 | DMSO | 10 | p38 MAPK | |
| | | 47-6 | | | | | | |
| B4 | EI-148 | 84477- | H-7 | 364.3 | DMSO | 10 | PKA, PKG, | |
| | | 87-2 | | | | | MLCK, and | |
| | | | | | | | PKC. | |
| B5 | EI-195 | 84468- | H-9 | 324.3 | DMSO | 10 | PKA, PKG, | |
| | | 17-7 | | | | | MLCK, and | |
| | | | | | | | PKC. | |
| B6 | EI-156 | 62996- | Staurosporine | 466.5 | DMSO | 10 | Pan- | |
| | | 74-1 | | | | | specific | |
| B7 | EI-228 | 133550- | AG-494 | 280.3 | DMSO | 10 | EGFRK, | |
| | | 35-5 | | | | | PDGFRK | |
| B8 | EI-267 | | AG-825 | 397.5 | DMSO | 10 | HER1-2 | |
| B9 | EI-185 | 125697- | Lavendustin A | 381.4 | DMSO | 10 | EGFRK | |
| | | 92-9 | | | | | | |
| B10 | EI-253 | 136831- | RG-14620 | 274.1 | DMSO | 10 | EGFRK | |
| | | 49-7 | | | | | | |
| B11 | EI-191 | 118409- | Tyrphostin 23 | 186.1 | DMSO | 10 | EGFRK | |
| | | 57-7 | | | | | | |
| B12 | EI-187 | 118409- | Tyrphostin 25 | 202.1 | DMSO | 10 | EGFRK | |
| | | 58-8 | | | | | | |
| C1 | EI-257 | 122520- | Tyrphostin 46 | 204.2 | DMSO | 10 | EGFRK, | |
| | | 85-8 | | | | | PDGFRK | |
| C2 | EI-188 | 122520- | Tyrphostin 47 | 220.2 | DMSO | 10 | EGFRK | |
| | | 86-9 | | | | | | |
| C3 | EI-189 | 122520- | Tyrphostin 51 | 268.2 | DMSO | 10 | EGFRK | |
| | | 90-5 | | | | | | |
| C4 | EI-190 | 2826- | Tyrphostin 1 | 184.2 | DMSO | 10 | Negative | |
| | | 26-8 | | | | | control for | |
| | | | | | | | tyrosine | |
| | | | | | | | kinase | |
| | | | | | | | inhibitors. | |
| C5 | EI-335 | 116313- | Tyrphostin AG 1288 | 231.2 | DMSO | 10 | Tyrosine | N |
| | | 73-6 | | | | | kinases | |
| C6 | EI-277 | 63177- | Tyrphostin AG 1478 | 315.8 | DMSO | 10 | EGFRK | |
| | | 57-1 | | | | | | |
| C7 | AC-1133 | 71897- | Tyrphostin AG 1295 | 234.3 | DMSO | 10 | Tyrosine | |
| | | 07-9 | | | | | kinases | |
| C8 | EI-215 | 10537- | Tyrphostin 9 | 282.4 | DMSO | 10 | PDGFRK | Y |
| | | 47-0 | | | | | | |
| C9 | EI-247 | | HNMPA (Hydroxy-2- | 238.2 | DMSO | 10 | IRK | |
| | | | naphthalenylmethylphosphonic | | | | | |
| | | | acid) | | | | | |
| C10 | EI-274 | 477-84-9 | Damnacanthal | 282.3 | DMSO | 10 | p56 lck | |
| C11 | EI-271 | 10083- | Piceatannol | 244.3 | DMSO | 10 | Syk | |
| | | 24-6 | | | | | | |
| C12 | EI-275 | 172889- | PP1 | 281.4 | DMSO | 10 | Src family | |
| | | 26-8 | | | | | | |
| D1 | EI-272 | 133550- | AG-490 | 294.3 | DMSO | 10 | JAK-2 | |
| | | 35-3 | | | | | | |
| D2 | EI-263 | | AG-126 | 215.2 | DMSO | 10 | IRAK | |
| D3 | EI-229 | | AG-370 | 259.3 | DMSO | 10 | PDGFRK | |
| D4 | EI-258 | | AG-879 | 316.5 | DMSO | 10 | NGFRK | |
| D5 | ST-420 | 154447- | LY 294002 | 307.4 | DMSO | 10 | PI 3-K | |
| | | 36-6 | | | | | | |
| D6 | ST-415 | 19545- | Wortmannin | 428.4 | DMSO | 10 | PI 3-K | |
| | | 26-7 | | | | | | |
| D7 | EI-246 | 133052- | GF 109203X | 412.5 | DMSO | 10 | PKC | |
| | | 90-1 | | | | | | |
| D8 | EI-226 | 548-04-9 | Hypericin | 504.4 | DMSO | 10 | PKC | |
| D9 | EI-283 | 138489- | Ro 31-8220 | 553.7 | DMSO | 10 | PKC | N |
| | | 18-6 | | | | | | |
| D10 | EI-155 | 123-78-4 | Sphingosine | 299.5 | DMSO | 10 | PKC | |
| D11 | EI-196 | 127243- | H-89 | 519.2 | DMSO | 10 | PKA | |
| | | 85-0 | | | | | | |
| D12 | EI-158 | 84478- | H-8 | 338.3 | DMSO | 10 | PKA, PKG | |
| | | 11-5 | | | | | | |
| E1 | EI-184 | 91742- | HA-1004 | 329.8 | DMSO | 10 | PKA, PKG | |
| | | 10-8 | | | | | | |
| E2 | EI-233 | 103745- | HA-1077 | 327.8 | DMSO | 10 | PKA, PKG | Y |
| | | 39-7 | | | | | | |
| E3 | EI-232 | | HDBA (2-Hydroxy-5-(2,5- | 275.3 | DMSO | 10 | EGFRK, | |
| | | | dihydroxybenzylamino)benzoic | | | | CaMK II | |
| | | | acid) | | | | | |
| E4 | EI-230 | 127191- | KN-62 | 721.9 | DMSO | 10 | CaMK II | Y |
| | | 97-3 | | | | | | |
| E5 | EI-268 | | KN-93 | 501 | DMSO | 10 | CaMK II | |
| E6 | EI-197 | 109376- | ML-7 | 452.7 | DMSO | 10 | MLCK | |
| | | 83-2 | | | | | | |
| E7 | EI-153 | 105637- | ML-9 | 361.3 | DMSO | 10 | MLCK | |
| | | 50-1 | | | | | | |
| E8 | CC-100 | 452-06-2 | 2-Aminopurine | 135.1 | DMSO | 10 | p58 | Y |
| | | | | | | | PITSLRE | |
| | | | | | | | beta1 | |
| E9 | CC-202 | 158982- | N9-Isopropyl-olomoucine | 326.4 | DMSO | 10 | CDK | |
| | | 15-1 | | | | | | |
| E10 | CC-200 | 101622- | Olomoucine | 298.3 | DMSO | 10 | CDK | |
| | | 51-9 | | | | | | |
| E11 | CC-201 | 101622- | iso-Olomoucine | 298.4 | DMSO | 10 | Negative | |
| | | 50-8 | | | | | control for | |
| | | | | | | | olomoucine. | |
| E12 | CC-205 | 186692- | Roscovitine | 354.5 | DMSO | 10 | CDK | |
| | | 46-6 | | | | | | |
| F1 | EI-293 | 24386- | 5-Iodotubercidin | 392.2 | DMSO | 10 | ERK2, | Y |
| | | 93-4 | | | | | adenosine | |
| | | | | | | | kinase, | |
| | | | | | | | CK1, CK2, | |
| F2 | EI-295 | 62004- | LFM-A13 | 360 | DMSO | 10 | BTK | |
| | | 35-7 | | | | | | |
| F3 | EI-294 | 152121- | SB-202190 | 331.3 | DMSO | 10 | p38 MAPK | Y |
| | | 30-7 | | | | | | |
| F4 | EI-297 | 172889- | PP2 | 301.8 | DMSO | 10 | Src family | |
| | | 27-9 | | | | | | |
| F5 | EI-298 | 208260- | ZM 336372 | 389.4 | DMSO | 10 | cRAF | N |
| | | 29-1 | | | | | | |
| F6 | EI-306 | 5812- | SU 4312 | 264.3 | DMSO | 10 | Flk1 | |
| | | 07-7 | | | | | | |
| F7 | EI-303 | 146535- | AG-1296 | 266.3 | DMSO | 10 | PDGFRK | Y |
| | | 11-7 | | | | | | |
| F8 | EI-307 | 220904- | GW 5074 | 520.9 | DMSO | 10 | cRAF | |
| | | 83-6 | | | | | | |
| F9 | AC-1121 | 6865- | Palmitoyl-DL-carnitine Cl | 436.1 | DMSO | 10 | PKC | Y |
| | | 14-1 | | | | | | |
| F10 | EI-270 | 82-08-6 | Rottlerin | 516.6 | DMSO | 10 | PKC delta | Y |
| F11 | EI-147 | 446-72-0 | Genistein | 270.2 | DMSO | 10 | Tyrosine | |
| | | | | | | | Kinases | |
| F12 | ST-110 | 486-66-8 | Daidzein | 254.2 | DMSO | 10 | Negative | |
| | | | | | | | control for | |
| | | | | | | | Genistein. | |
| G1 | EI-146 | 63177- | Erbstatin analog | 194 | DMSO | 10 | EGFRK | Y |
| | | 57-1 | | | | | | |
| G2 | AC-1142 | 6151-25-3 | Quercetin dihydrate | 338.3 | DMSO | 10 | PI 3-K | |
| G3 | AC-1293 | | SU1498 | 390.5 | DMSO | 10 | Flk1 | |
| G4 | AC-1294 | 4452-06-6 | ZM 449829 | 182.2 | DMSO | 10 | JAK-3 | |
| G5 | EI-278 | 195462- | BAY 11-7082 | 207.3 | DMSO | 10 | IKK | N |
| | | 67-7 | | | | | pathway | |
| G6 | EI-231 | 53-85-0 | DRB (5,6-Dichloro-1-β-D- | 319.1 | DMSO | 10 | CK II | |
| | | | ribofuranosylbenzimidazole) | | | | | |
| G7 | EI-273 | | HBDDE (2,2′,3,3′,4,4′- | 338.4 | DMSO | 10 | PKC alpha, | |
| | | | Hexahydroxy-1,1′-biphenyl- | | | | PKC | |
| | | | 6,6′-dimethanol dimethyl ether) | | | | gamma | |
| G8 | EI-305 | 129-56-6 | SP 600125 | 220.2 | DMSO | 10 | JNK | |
| G9 | CC-206 | 479-41-4 | Indirubin | 262 | DMSO | 10 | GSK-3beta, | |
| | | | | | | | CDK5 | |
| G10 | CC-207 | 160807- | Indirubin-3′-monoxime | 277.3 | DMSO | 10 | GSK-3beta | N |
| | | 49-8 | | | | | | |
| G11 | EI-299 | 146986- | Y-27632 | 338.3 | DMSO | 10 | ROCK | |
| | | 50-7 | | | | | | |
| G12 | EI-310 | 142273- | Kenpaullone | 327.2 | DMSO | 10 | GSK-3beta | |
| | | 20-9 | | | | | | |
| H1 | EI-328 | 121-40-4 | Terreic acid | 154.1 | DMSO | 10 | BTK | Y |
| H2 | EI-332 | 35943- | Triciribine | 320.3 | DMSO | 10 | Akt | Y |
| | | 35-2 | | | | | signaling | |
| | | | | | | | pathway | |
| H3 | EI-336 | | BML-257 | 326.4 | DMSO | 10 | Akt | |
| H4 | EI-343 | | SC-514 | 224.3 | DMSO | 10 | IKK2 | |
| H5 | EI-344 | | BML-259 | 260.4 | DMSO | 10 | Cdk5/p25 | |
| H6 | EI-345 | 520-36-5 | Apigenin | 270.2 | DMSO | 10 | CK-II | |
| H7 | EI-346 | | BML-265 (Erlotinib analog) | 305.4 | DMSO | 10 | EGFRK | N |
| H8 | A-275 | 53123- | Rapamycin | 914.2 | DMSO | 10 | mTOR | Y |
| | | 88-9 | | | | | | |

TABLE 6

| | | |
|---|---|---|
| 5-Iodotubercidin | MAP kinase ERK2 (Ki = 530 nM), adenosine kinase (Ki = | casein kinase I and PKA, insulin receptor kinase |
| | 30 nM) | fragment (IC50 values range from 0.4-28 μM) |
| AG 1296 | PDGF receptor kinase (human PDGF a-receptors IC50 = | c-kit (80% inhibition at 5 μM) |
| | 1.0 μM and b-receptors IC50 = 800 nM) | |
| BAY 11-7082 | TNF-a-inducible phosphorylation of IkBa (IC50 = 10 μM) | |
| BML-265 (Erlotinib | | |
| analog) | | |
| HA-1077 | protein kinase A (Ki = 1.6 μM), protein kinase G (Ki = 1.6 | Rho-associated kinase (ROCK; IC50 = 10.7 μM) |
| | μM), and myosin light chain kinase (Ki = 36 μM) | |
| Indirubin-3′- | GSK-3b (glycogen synthase kinase 3b), Cdk1 (cyclin- | |
| monoxime | dependent kinase1) and Cdk5 (IC50 = 22 nM, 180 nM, and | |
| | 100 nM, respectively) | |
| KN-62 | CaM kinase II (Ki = 900 nM for rat brain CaM kinase II) | |
| KN-93 | CaM kinase II (Ki = 370 nM) | |
| Rapamycin | p70 S6 kinase (IC50 = 50 pM) | |
| Ro-31-8220 | protein kinase C (PKC; IC50 = 10 nM) over CaM kinase II | GSK-3 in primary adipocytes (IC50 = 6.8 nM) and in |
| | (IC50 = 17 μM) and protein kinase A (IC50 = 900 nM) | GSK-3b immunoprecipitates (IC50 = 2.8 nM) |
| Rottlerin | PKCd (IC50 = 3-6 μM) and PKCq; PKCa, PKCb, and PKCg | Has reduced inhibitory activity on PKCe, PKCh, and |
| SB 202190 | isoforms (IC50 = 30-42 μM); CaM kinase III (IC50 = 5.3 μM) | PKCz (IC50 = 80-100 μM) |
| | p38MAPK | |
| Akt Inhibitor V, | Akt1/2/3; preferentially induces apoptosis and growth arrest | |
| Triciribine | in cancer cells with aberrant Akt activity both in vitro (≧60% | |
| | in cell proliferation at 20 μM) and in vivo (≧80% inhibition in | |
| | tumor growth in mice at 1 mg/kg/day, i.p.) | |
| AG 17 | platelet-derived growth factor receptor tyrosine kinase | |
| | (IC50 = 500 nM) | |
| U0126 | A potent and specific inhibitor of MEK1 (IC50 = 72 nM) and | |
| | MEK2 (IC50 = 58 nM) | |
| ZM 336372 | c-Raf (IC50 = 70 nM). Inhibits c-Raf with ten-fold increased | |
| | potency compared to B-Raf, but does not inhibit many other | |
| | protein kinases (even at 50 μM) with the exception of | |
| | SAPK2a/p38a (IC50 = 2 μM) and SAPK2b/p38b2 (IC50 = 2 μM) | |

Evidences:
p38MAPK or c-raf or PKA/ROCK inhibition leads to enhanced proliferation
PKC inhibition decreases proliferation
p70S6K inhibition decreases proliferation
Akt inhibition decreases proliferation
MEK1 inhibition decreases proliferation

TABLE 7

| | | | | |
|---|---|---|---|---|
| [nM] | | | | |
| log(inhibitor) vs. | | | | |
| response | CTSC#1.1 | CTSC#18 | CTSC#CRO-I | CTSC#85 |

Best-fit values

| | | | | |
|---|---|---|---|---|
| BOTTOM | 70838 | 48695 | 74934 | 74515 |
| TOP | 128211 | 180029 | 189188 | 90863 |
| LOGIC50 | 2.07 | 2.402 | 1.67 | 2.08 |
| IC50 | 117.4 | 252.1 | 46.74 | 120.3 |
| Span | 57373 | 131334 | 114255 | 16348 |

Std. Error

| | | | | |
|---|---|---|---|---|
| BOTTOM | 2309 | 5728 | 3038 | 2214 |
| TOP | 3025 | 5081 | 5268 | 2871 |
| LOGIC50 | 0.09403 | 0.08533 | 0.07797 | 0.3148 |
| Span | 3432 | 6448 | 5792 | 3263 |

95% Confidence Intervals

| | | | | |
|---|---|---|---|---|
| BOTTOM | 66157 to 75518 | 37083 to 60306 | 68775 to 81093 | 70027 to 79003 |
| TOP | 122079 to 134343 | 169727 to 190330 | 178509 to 199867 | 85042 to 96684 |
| LOGIC50 | 1.879 to 2.260 | 2.229 to 2.575 | 1.512 to 1.828 | 1.442 to 2.718 |
| IC50 | 75.69 to 182.1 | 169.3 to 375.4 | 32.48 to 67.26 | 27.68 to 523.0 |
| Span | 50416 to 64330 | 118262 to 144405 | 102512 to 125997 | 9734 to 22962 |

Goodness of Fit

| | | | | |
|---|---|---|---|---|
| Degrees of | 37 | 37 | 37 | 37 |
| Freedom | | | | |
| R² | 0.884 | 0.9199 | 0.9145 | 0.4061 |
| Absolute Sum | 1.74E+09 | 6.03E+09 | 4.60E+09 | 1.58E+09 |
| of Squares | | | | |
| Sy.x | 6859 | 12767 | 11153 | 6527 |

Number of points analyzed

| | | | |
|---|---|---|---|
| 40 | 40 | 40 | 40 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method for identifying a metabolic target in a cancer stem cell, comprising using a protein microarray to identify intracellular signaling networks within a population of cancer stem cells that respond to a growth factor for the stem cell.

2. The method of claim 1, wherein the protein microarray is a reverse phase protein microarray.

3. The method of claim 1, wherein the growth factor is EGF.

4. The method of claim 3, wherein the EGF is capable of activating an EGF Receptor (EGFR).

5. The method of claim 1, wherein the population of cancer stem cells is sampled from a patient or subject diagnosed with cancer or from a patient or subject who has not previously been diagnosed with cancer, as part of a cancer screening program.

6. The method of claim 1, wherein having identified the metabolic target, suitable treatments for a patient are identified and the treatments administered to the patient, so as to provide a personalized treatment program, specific for the patient, based on the metabolic target.

7. The method of claim 1, wherein once a particular target or pattern of targets has been identified by the identification of the signaling networks, the pattern of targets is cross-referenced with a database of targets or target patterns.

8. A method of treating a patient with cancer by identifying a metabolic target in a cancer stem cell, the method comprising:
using a protein microarray to identify intracellular signaling networks within a population of cancer stem cells that respond to a growth factor for the stem cell; and,
treating the cancer by administering a therapeutic agent directed at said metabolic target.

9. A method of determining a personalized therapeutic regime, the method comprising:
receiving metabolic information relating to a cancer stem cell from a patient;
determining at least one metabolic target criteria in said cancer stem cell;
receiving personal information relating to the patient;
determining personal criteria relevant to the personalized therapeutic regime using the personal information; and,
combining the at least one metabolic target criteria and the personal criteria to determine the personalized therapeutic regime for the patient.

10-23. (canceled)

24. The method of claim 1, wherein the cancer stem cells show EGF-R activation:
wherein the cancer stem cells show Bcl-2 hyper-phosphorylation;
wherein the cancer stem cells show hyper-phosphorylated p38MAPK, NF-κB, and Shc, which is indicative of Melanoma cancer stem cells:
wherein the cancer stem cells show enhanced HER2 signaling which is indicative of colon cancer stem cells;
wherein the cancer stem cells show mTOR pathway hyper-activation or hyper-phosphorylated Bad levels which is indicative of lung cancer stem cells; or,
wherein the cancer stem cells show both mTOR and EGF-R hyper-phosphorylation or low EGF-R and high mTOR and GSK3-betalevels, or high phospho-Bad and phospho-Adducin levels which is indicative of Glioblastoma cancer stem cells.

25-29. (canceled)

30. A method for identifying a metabolic target in a colon cancer stem cell, comprising using a protein microarray to identify intracellular signaling networks within a population of cancer stem cells that respond to a growth factor for the stem cell.

31. A method of facilitating proliferation of colon cancer stem cells comprising contacting said cells with one or more inhibitors of p38MAPK, c-Raf or PKA/ROCK.

32. A method of reducing the number of colon cancer stem cells in a population of said cells, comprising contacting said cells with geldanamycin 17-aag or 17-dmag and, optionally, with one or more inhibitors of inhibitors of PKC, p70S6K, Akt and/or MEK1.

33. A method of treating colon cancer in a patient, comprising administering geldanamycin17-aag or 17-dmagand, optionally, one or more inhibitors of p38MAPK, c-Raf and/or PKA/ROCK to said patient.

34. A diagnostic test for establishing a personalized treatment regime for a colon cancer patient, comprising:
i. conducting microarray analysis on cancer stem cells to predict pathway activation linked to colon cancer;
ii. probing isolated colon cancer stem cells with one or more inhibitors, to validate a predicted pathway activation; and
iii. combining pathway activation data with inhibitor data to determine indvidualized therapies for said patient.
